(19) United States
(12) Patent Application Publication
(10) Pub. No.: US 2024/0270756 A1
(43) Pub. Date: Aug. 15, 2024

(54) Integrase Inhibitor and Use Thereof

(71) Applicant: Ascletis BioScience Co., Ltd., Hangzhou (CN)

(72) Inventors: Jinzi Jason WU, Hangzhou (CN); Bin LIANG, Hangzhou (CN)

(21) Appl. No.: 18/416,152

(22) Filed: Jan. 18, 2024

Related U.S. Application Data
(60) Provisional application No. 63/480,419, filed on Jan. 18, 2023.
(60) Provisional application No. 63/530,876, filed on Aug. 4, 2023.
(60) Provisional application No. 63/531,160, filed on Aug. 7, 2023.
(60) Provisional application No. 63/546,011, filed on Oct. 27, 2023.

(30) Foreign Application Priority Data
Jul. 12, 2023 (CN) 202310851935.X
Jul. 31, 2023 (CN) 202310953344.3
Aug. 1, 2023 (CN) 202310960689.1
Oct. 25, 2023 (CN) 202311395259.6

(51) Int. Cl. C07D 498/14; A61K 31/4985; A61K 31/5383

(52) U.S. Cl. CPC C07D 498/14; A61K 31/4985; A61K 31/5383

(57) ABSTRACT

Disclosed are a compound of Formula I, stereoisomers, pharmaceutically acceptable salts and deuterated compounds thereof and a method for treating, inhibiting and/or preventing a disease or disorder in a subject in need thereof:

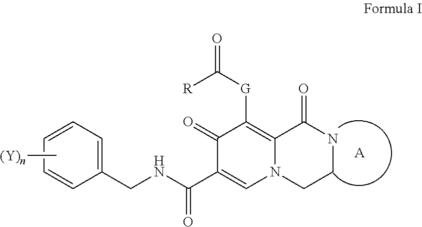

wherein A, R, G, Y and n are defined as in the present disclosure.

### CROSS-REFERENCE TO RELATED APPLICATIONS

This present application claims the benefit of priorities to follows patent applications: the Chinese patent application No. CN202310851935.X filed on Jul. 12, 2023, the Chinese patent application No. CN 202310960689.1 filed on Aug. 1, 2023, the Chinese patent application No. CN 202310953344.3 filed on Jul. 31, 2023, the Chinese patent application No. CN 202311395259.6 filed on Oct. 25, 2023, the America patent application No. U.S. 63/480,419 filed on Jan. 18, 2023, the America patent application No. U.S. 63/530,876 filed on Aug. 4, 2023, the America patent application No. U.S. 63/531,160 filed on Aug. 7, 2023, the America patent application No. U.S. 63/546,011 filed on Oct. 27, 2023, the aforementioned patents hereby incorporated by reference in their entirety.

### FIELD

The present disclosure relates to the field of medicine, and more particularly, to prodrugs of integrase inhibitors and use thereof.

### BACKGROUND

Significant advances have been made in the development of effective diagnosis and treatment of human immunodeficiency virus type 1 (HIV-1). Antiretroviral therapy (ART) significantly reduces disease-associated incidence rate and mortality rate, resulting in a near-normal quality of life for infected individuals. However, ART requires lifelong treatment in order to inhibit viral replication and prevent the onset of AIDS. In addition, the efficacy of ART may be hindered by HIV-1 resistance, drug toxicity and poor patient compliance. Treatment of fatigue, lack of economic and social support, coexisting psychiatric symptoms, and/or drug abuse may lead to failure to comply with key ART regimens. Dolutegravir (DTG), cabotegravir (CAB) and bictegravir (BTG) are a class of integrase inhibitors or integrase chain transfer inhibitors (INSTIs).

### SUMMARY

In one aspect, the present disclosure relates to a compound of Formula I, stereoisomers, pharmaceutically acceptable salts and deuterated compounds thereof:

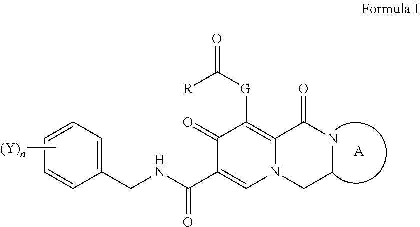

Wherein,
A is selected from the group consisting of

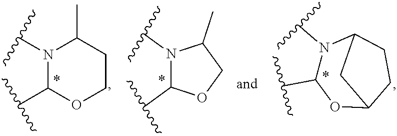

wherein * represents an R or S configuration;
n is 0, 1, 2 or 3;
G is independently selected from oxygen or Gr;
Wherein the Gr is

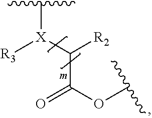

Wherein R₂ and R₃ are each independently selected from the group consisting of hydrogen, hydroxy, cyano, C₁₋₆ alkyl, C₆₋₁₀ aryl, C₃₋₁₀ cycloalkyl, arylheterocyclyl, C₁₋₆ alkyl-C₆₋₁₀ aryl, C₁₋₆ alkyl-arylheterocyclyl and C₁₋₆ alkyl-C₃₋₁₀ cycloalkyl;
Or the R₂ and the R₃ may be combined with an atom to which they are attached to form a 4- to 10-membered heterocyclyl; wherein heterocyclyl may be substituted with 1 to 4 R₄;
The R₄ is independently selected from the group consisting of hydrogen, halogen, C₁₋₆ alkyl and alkoxy;
Or any two R₄ may be combined with an atom to which they are attached to form a C₃₋₁₀ cycloalkyl; wherein cycloalkyl may be substituted with 1 to 4 hydrogen, halogen or C₁₋₆ alkyl;
X is selected from the group consisting of oxygen and nitrogen;
Y is halogen;
m is 0 to 20;
R is independently selected from the group consisting of C₂₋₃₀ saturated or unsaturated hydrocarbyl and aliphatic group, wherein hydrocarbyl and aliphatic group may be optionally substituted with at least one heteroatom selected from the group consisting of N, O and S;
The R may be optionally substituted with a substituent selected from the group consisting of halo, carboxy, sulfonyl, —S═O, —S(═O)₂, aryl, heteroaryl, C₃₋₁₀ saturated or unsaturated carbocycle and C₃₋₁₀ heterocyclyl, wherein the aryl, heteroaryl, carbocycle and heterocyclyl were optionally substituted with H, C₁₋₂₀ alkyl, C₂₋₂₀ alkenyl, C₂₋₂₀ alkynyl, C₁₋₂₀ haloalkyl, C₂₋₂₀ haloalkenyl, C₂₋₂₀ haloalkynyl, C₀₋₂₀ alkyl-C₆₋₁₀ aryl, heterocyclyl, C₀₋₂₀ alkyl-C₃₋₁₀ cycloalkyl, C₀₋₂₀ alkyl-O—C₁₋₆ alkyl and C₀₋₂₀ alkyl-O—C₁₋₆ haloalkyl.

In another aspect, the present application relates to a composition comprising a compound of formula I, stereoisomers, pharmaceutically acceptable salts or deuterated compounds thereof of the present application.

In another aspect, the present disclosure relates to a method for treating, inhibiting and/or preventing a disease or disorder in a subject in need thereof, comprising administering an effective amount of the compound of Formula I, a stereoisomer, a pharmaceutically acceptable salt or a deuterated compound thereof of the present disclosure to the subject.

### BRIEF DESCRIPTION OF THE DRAWINGS

FIG. 1 shows Plasma Concentration-Time Curve (AUC) after subcutaneous injection of the compounds of the present disclosure.

FIG. 2 shows concentrations of active ingredients in plasma after compound administration.

FIG. 3 shows after a single subcutaneous injection of Compound 13 and Compound 21 (dosage: equivalent to 18 mg/kg of bictegravir) in rats, correlation of the mean plasma concentrations of the corresponding active ingredient (bictegravir) versus with time.

FIG. 4 shows after a single subcutaneous injection of Compound 14 (dosage: equivalent to 18 mg/kg of cabotegravir) in rats, correlation of the mean plasma concentrations of the corresponding active ingredient (cabotegravir) versus with time.

### DETAILED DESCRIPTION

In the following description, certain specific details are included to provide a thorough understanding of the various disclosed embodiments. However, those skilled in the relevant art will recognize that embodiments may be practiced without one or more of these specific details, but with other methods, components, materials, and the like.

Unless otherwise claimed in this disclosure, throughout the specification and the claims that follow, the words “comprise” and “comprising” are to be construed in an open, inclusive sense, i.e., “including, but not limited to”.

As used in the present disclosure and the appended claims, the singular reference without an indication of quantity includes the plural reference unless the context clearly dictates otherwise.

Reference throughout this specification to “one embodiment” or “an embodiment” or “in another embodiment” or “in some embodiments” is meant to include in at least one embodiment specific reference elements, structures, or features relevant to those described in that embodiment. Thus, the appearances of the phrases “in one embodiment” or “in an embodiment” or “in another embodiment” or “in some embodiments” in various places throughout the specification are not necessarily all referring to the same embodiment. Furthermore, the specific elements, structures, or features may be combined in any suitable manner in one or more embodiments.

It is to be understood that the singular form used in the specification of the present disclosure and the appended claims include plural referents unless the context clearly dictates otherwise. Thus, for example, reference to a sustained release tablet comprising “a pharmaceutically acceptable excipient” includes one pharmaceutically acceptable excipient, or two or more pharmaceutically acceptable excipients.

### Definition

Unless defined otherwise, all technical and scientific terms used herein have the same meaning as commonly understood by one of ordinary skill in the art. The dashes at the front or end of the chemical group are for convenience to indicate the point of attachment to the parent molecule, Chemical groups may be described with or without one or more dashes without losing their ordinary meaning. A prefix such as “Cᵤ₋ᵥ” or “Cᵤ-Cᵥ” indicates that the following groups have from u to v carbon atoms, where u and v are integers. For example, “C₁₋₆ alkyl” or “C₁-C₆ alkyl” means that the alkyl group has 1 to 6 carbon atoms.

“Alkyl” is a monovalent or divalent linear or branched saturated hydrocarbon group. For example, the alkyl group may have 1 to 10 carbon atoms (i.e., C₁₋₁₀ alkyl) or 1 to 8 carbon atoms (i.e., C₁₋₈ alkyl) or 1 to 6 carbon atoms (i.e., C₁₋₆alkyl) or 1 to 4 carbon atoms (i.e., C₁₋₄alkyl). Examples of alkyl groups include, but are not limited to, methyl (Me, —CH₃), ethyl (Et, —CH₂CH₃), 1-propyl (n-Pr, n-propyl, CH₂CH₂CH₃), 2-propyl (i-Pr, i-Propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₃), 2-pentyl (—CH(CH₃)CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₃), 2-hexyl (—CH(CH₃)CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃), and octyl (—(CH₂)₇CH₃). Alkyl groups may be unsubstituted or substituted.

“Alkenyl” is a monovalent or divalent linear or branched hydrocarbon group having at least one carbon-carbon double bond. For example, an alkenyl group may have 2 to 8 carbon atoms (i.e., C₂₋₈ alkenyl) or 2 to 6 carbon atoms (i.e., C₂₋₆ alkenyl) or 2 to 4 carbon atoms (i.e., C₂₋₄ alkenyl). Examples of alkenyl groups include, but are not limited to, —CH═CH₂, —CH₂CH═CH₂, and —CH₂—CH═CH—CH₃. Alkenyl groups may be unsubstituted or substituted.

“Alkynyl” is a monovalent or divalent linear or branched hydrocarbon group having at least one carbon-carbon triple bond. For example, an alkynyl group may have 2 to 8 carbon atoms (i.e., C₂₋₈ alkynyl) or 2 to 6 carbon atoms (i.e., C₂₋₆ alkynyl) or 2 to 4 carbon atoms (i.e., C₂₋₄ alkynyl). Examples of alkynyl include, but are not limited to, —C≡CH, —CH₂C≡CH, and —CH₂—C≡C—CH₃. Alkynyl groups may be unsubstituted or substituted.

“Alkoxyalkyl” is an alkoxy group attached to an alkyl group as defined above such that the alkyl group is bivalent. For example, C₂₋₆ alkoxyalkyl includes —CH₂—OMe, —CH₂—O-iPr, —CH₂—CH₂—OMe, —CH₂—CH₂—O—CH₂—CH₃, and —CH₂—CH₂—O-tBu. Alkoxyalkyl groups may be unsubstituted or substituted.

“Halogen” refers to fluorine (—F), chlorine (—Cl), bromine (—Br) and iodine (—I).

“Haloalkyl” is an alkyl group as defined herein, wherein one or more hydrogen atoms of the alkyl group are independently substituted with one halogen, which halogen may be the same or different, such that the alkyl group is divalent. Alkyl and halogen may be any of those described above. In some embodiments, haloalkyl determines the number of carbon atoms of the alkyl moiety, for example, C₁₋₄haloalkyl includes CF₃, CH₂F, CHF₂, CH₂CF₃, CH₂CH₂CF₃, CCl₂CH₂CH₃, and C(CH₃)₂(CF₂H). Haloalkyl groups may be unsubstituted or substituted.

“Aryl” refers to a monovalent or divalent mono- or polycondensed all-carbon aromatic ring system in which the ring is aromatic. For example, in some embodiments, one aryl group has 6 to 20 carbon atoms, 6 to 14 carbon atoms, 6 to 12 carbon atoms, or 6 to 10 carbon atoms. Aryl groups include one phenyl group. Aryl also includes a plurality of condensed ring systems (e.g., ring systems consisting of 2, 3, or 4 rings) having about 9 to 20 carbon atoms, wherein the plurality of rings are aromatic. Where valence requirements permit, the rings of multiple condensed ring systems may be interconnected by fused bonds. It will also be appreciated that when referring to a member aryl group (such as an aryl group of 6 to 10 members) of an atomic range, the atomic range refers to the total ring atoms of the aryl group. For example, six member aryl groups include phenyl and ten member aryl groups include naphthyl. Non-limiting examples of aryl groups include, but are not limited to, phenyl, naphthyl, anthracenyl, and the like. Aryl groups may be unsubstituted or substituted.

“Alkylaryl” refers to an alkyl group, as defined herein, wherein one or more hydrogen atoms of the alkyl group are independently substituted with one aryl group, in which the aryl group may be the same or different. The alkyl and aryl groups may be any of those described above. Such an alkyl groups is divalent. In some embodiments, alkylaryl group has 7 to 24 carbon atoms, 7 to 16 carbon atoms, 7 to 13 carbon atoms, or 7 to 11 carbon atoms. Alkylaryl, as defined by the number of carbon atoms, refers to the total number of carbon atoms present in the alkyl and aryl groups. For example, C₇ alkylaryl refers to benzyl, while C₁₁ alkylaryl includes 1-methylnaphthalene and n-pentylphenyl. In some embodiments, the number of carbon atoms of the alkyl and aryl moieties may be designated separately, e.g., C₁₋₆ alkyl-C₆₋₁₀ aryl. Non-limiting examples of alkylaryl include, but are not limited to, benzyl, 2,2-dimethylphenyl, n-pentylphenyl, I-methylnaphthyl, 2-ethylnaphthyl and the like. Alkylaryl groups may be unsubstituted or substituted.

“5- to 10-membered aromatic heterocycle” or “heteroaromatic ring” refers to a single aromatic ring having an atom other than at least one carbon in the ring, wherein the atom is selected from the group consisting of oxygen, nitrogen, and sulfur; “Heteroaryl” also includes a plurality of condensed ring systems having at least one such aromatic ring, the plurality of condensed ring systems being further described below. Thus, “heteroaryl” includes monoaromatic rings of about 1 to 6 carbon atoms and about 1 to 4 heteroatoms selected from the group consisting of oxygen, nitrogen and sulfur. Sulfur and nitrogen atoms may also be present in oxidized form as long as the ring is aromatic. Exemplary heteroaryl ring systems include, but are not limited to, pyridyl, pyrimidinyl, oxazolyl, or furyl. “Heteroaryl” also includes a plurality of condensed ring systems (e.g., ring systems consisting of 2, 3, or 4 rings) in which a heteroaryl as defined above is condensed with one or more rings selected from the group consisting of heteroaryl (to form, e.g., 1, 8-naphthyridinyl) and aryl (to form, e.g., benzimidazolyl or indazolyl) to form a plurality of condensed ring systems. Thus, a heteroaryl group (a single aromatic ring or multiple condensed ring systems) can have about 1 to 20 carbon atoms and about 1 to 6 heteroatoms within the heteroaryl ring. For example, tetrazolyl has 1 carbon atom and 4 nitrogen heteroatoms in the ring. Where valence requirements permit, the rings of multiple condensed ring systems may be interconnected by fused bonds. It will be appreciated that each ring of a plurality of condensed ring systems may be interconnected in any order. It should be understood that the point of attachment of the heteroaryl or heteroaryl multiple condensation ring system can be any suitable atom of the heteroaryl or heteroaryl multiple condensation ring system, including carbon atoms and heteroatoms (e.g., nitrogen). It is also to be understood that when reference is made to a member heteroaryl group (e.g., a 5 to 10 member heteroaryl group) of an atomic range, the atomic range is for the total ring atoms of the heteroaryl group, including carbon atoms and heteroatoms. It is also to be understood that rings of multiple condensed ring systems may include aryl rings fused to a heterocyclic ring having a saturated or partially unsaturated bond (e.g., a 3-, 4-, 5-, 6-, or 7-membered ring) having about 1 to 6 cyclic carbon atoms and about 1 to 3 cyclic heteroatoms selected from oxygen, nitrogen, and sulfur in the ring. For example, 5-membered heteroaryl includes thiazolyl and 10-membered heteroaryl includes quinolinyl. Exemplary heteroaryl groups include, but are not limited to, pyridyl, pyrrolyl, pyrazinyl, pyrimidinyl, pyrazolyl, thienyl, indolyl, imidazolyl, oxazolyl, isoxazolyl, thiazolyl, furanyl, oxadiazolyl, thiazolyl, quinolinyl, isoquinolinyl, benzothiazolyl, benzoxazolyl, indazolyl, quinoxazolyl, quinazolyl, benzofuranyl, benzimidazolyl, thiophenyl, pyrrolo [2,3-b]pyridyl, quinazolinyl-4(3H)-one, triazolyl, and tetrazolyl. Heteroaryl groups may be unsubstituted or substituted.

“Cycloalkyl” is a monovalent or divalent single all-carbocyclic ring or multiple condensed all-carbocyclic ring systems, wherein the ring in each example is a non-aromatic saturated or unsaturated ring. For example, in some embodiments, a cycloalkyl group has 3 to 12 carbon atoms, 3 to 10 carbon atoms, 3 to 8 carbon atoms, 3 to 6 carbon atoms, 3 to 5 carbon atoms, or 3 to 4 carbon atoms. Exemplary monocyclic cycloalkyl groups include cyclopropyl, cyclobutyl, cyclopentyl, cyclohexyl, cycloalkenyl, cycloheptyl, cycloheptenyl, and cyclooctyl. Cycloalkyl also includes multiple condensed ring systems having about 7 to 12 carbon atoms (e.g., ring systems including 2 rings). Where valence requirements permit, the rings of multiple condensed ring systems may be interconnected by a fusion bond, a spiral bond, or a bridging bond. Exemplary polycyclic cycloalkyl groups include octahydropentene, bicyclic[2.2.1]heptane, bicyclo[2.2.2]octane, bicyclo[2.2]oct-2-ene and spiro[2.5]octane. The cycloalkyl group may be unsubstituted or substituted.

As used herein, “heterocycle” or “heterocycle” or “heterocyclyl group” refers to a single saturated or partially unsaturated non-aromatic ring or non-aromatic polycyclic ring system having at least one heteroatom in the ring (i.e., at least one cyclic (i.e., cyclic) heteroatom selected from oxygen, nitrogen, and sulfur). Unless otherwise specified, heterocyclic groups have from 3 to about 20 cyclic atoms, e.g., from 3 to 12 cyclic atoms, e.g., from 4 to 12 cyclic atoms, from 4 to 10 cyclic atoms, or from 3 to 8 cyclic atoms, or from 3 to 6 cyclic atoms, or from 4 to 6 cyclic atoms, or from 4 to 5 cyclic atoms. Thus, the term includes single saturated or partially unsaturated rings (e.g., 3, 4, 5, 6, or 7-membered rings) having about 1 to 6 cyclic carbon atoms and about 1 to 3 cyclic heteroatoms, the heteroatoms in these rings being selected from the group consisting of oxygen, nitrogen, and sulfur. Where valence requirements permit, the rings of multiple condensed rings, such as bicyclic heterocycles, may be interconnected by fused bonds, spiral bonds, and bridged bonds. Heterocycles include, but are not limited to, azacycle, aziridine, imidazolidine, morpholine, oxirane, oxirane, thiacycle, piperazine, piperidine. Pyrazolidine, piperidine, pyrrolidine, pyrrolidone, tetrahydrofuran, tetrahydrothiophene, dihydropyridine, tetrahydropyridine, quinuclidine, 2-oxo-6-azaspiro[3. 3]heptan-6-yl, 6-oxa-1-azaspiro[3.3]heptan-1-yl, 2-thia-6-azaspiro[3.3]heptan-6-yl, 2,6-diazaspiro[3.3]heptan-2-yl, 2-azabicyclo[3.1.0]hex-2-yl, 3-azabicyclo[3.0]hexyl, 2-azabicyclo[2.1.1]hexyl, 2-azabicyclo[2.2.1]hept-2-yl, 4-azaspiro[2.4]heptyl, 5-azaspiro[2.4]heptyl, and the like. Heterocyclyl groups may be unsubstituted or substituted.

“Alkylheteroaryl” refers to an alkyl group, as defined herein, wherein one or more hydrogen atoms of the alkyl group are independently substituted with a heteroaryl group, in which the heteroaryl group may be the same or different, such that the alkyl group is divalent. The alkyl and heteroaryl groups may be any of the above. In some embodiments, the number of atoms of the alkyl and heteroaryl moieties can be designated separately, e.g., C₁₋₆ alkyl-5 to 10-membered heteroaryl having 1 to 4 heteroatoms, in which each heteroatom is independently N, O, or S. Alkylheteroaryl group may be unsubstituted or substituted.

“Oxo” refers to ═O.

As used herein, “substituted” means wherein one or more hydrogen atoms of a group are independently substituted with one or more substituents (e.g., 1, 2, 3, or 4 or more).

“Compounds of the present disclosure” include compounds disclosed herein, e.g., compounds of the present disclosure include compounds of Formula I, including compounds of the Examples. In some embodiments, “compounds of the present disclosure” include compounds of Formula I.

“Pharmaceutically acceptable excipients” include, but are not limited to, any adjuvants, carriers, excipients, lubricants, sweeteners, diluents, preservatives, dyes/colorants, flavoring agents, surfactants, wetting agents, dispersing agents, suspending agents, stabilizers, isotonic agents, solvents or emulsifiers, which have been approved by the U.S. Food and Drug Administration for use in humans or livestock.

As used herein, a “therapeutically effective amount” or “effective amount” refers to an amount that is effective to elicit a desired biological or medical response, including an amount of a compound that, when administered to a subject for treating a disease, is sufficient to affect the treatment of the disease. The effective amount will vary depending on the compound, the disease and its severity, as well as the age, weight, and other factors of the subject to be treated. An effective amount may include a range of amounts. As understood in the art, an effective amount may be one or more doses, i.e., one or more doses may be required to achieve the desired therapeutic end point. An effective amount can be considered in the case of administration of one or more therapeutic agents, and a single agent can be considered to be administered in an effective amount if a desired or beneficial result is or has been achieved with one or more other agents. Due to the combined effects of the compounds (e.g., additive or synergistic effects), the appropriate dosage of any co-administered compound may be selectively reduced.

As used herein, “co-administration” means administration of a unit dose of a compound of the present disclosure before or after administration of a unit dose of one or more additional therapeutic agents, e.g., administration of a compound of the present disclosure within a few seconds, minutes, or hours of administration of one or more additional therapeutic agents. For example, in some embodiments, a unit dose of a compound of the present disclosure is administered first, followed by a unit dose of one or more additional therapeutic agents within a few seconds or minutes. Alternatively, in other embodiments, a unit dose of one or more additional therapeutic agents is administered first, followed by a unit dose of a compound of the present disclosure within seconds or minutes. In some embodiments, a unit dose of a compound of the present disclosure is administered first, followed by a unit dose of one or more additional therapeutic agents after a few hours (e.g., 1-12 hours). In other embodiments, a unit dose of one or more additional therapeutic agents is administered first, followed by a unit dose of a compound of the present disclosure after a few hours (e.g., 1-12 hours). Co-administration of a compound disclosed herein with one or more additional therapeutic agents generally refers to simultaneous or sequential administration of a compound disclosed herein and one or more additional therapeutic agents such that a therapeutically effective amount of each agent is present in the subject's body.

Also provided are pharmaceutically acceptable salts, hydrates, solvates, isomeric forms, polymorphs, and prodrugs of the compounds described herein.

“Pharmaceutically acceptable” or “physiologically acceptable” refers to compounds, salts, compositions, dosage forms and other materials useful in the preparation of pharmaceutical compositions suitable for veterinary or human pharmaceutical use.

The compounds described herein may be prepared and/or formulated as pharmaceutically acceptable salts or, where appropriate, as free bases. A pharmaceutically acceptable salt is a non-toxic salt of a free base form of a compound that has the desired pharmacological activity of the free base. These salts may be extracted from inorganic or organic acids or bases. For example, a compound containing a basic nitrogen can be prepared as a pharmaceutically acceptable salt by contacting the compound with an inorganic or organic acid. Non-limiting examples of pharmaceutically acceptable salts include sulfates, pyrosulfates, disulfates, sulfites, disulfates, phosphates, monohydrogenphosphates, dihydrogenphosphates, metaphosphates, pyrophosphates, chloride, bromide, iodide, acetate, propionate, decanoate, octanoate, acrylate, formate, isobutyrate, hexanoate, heptanoate, propoxide, oxalate, malonate, ferrite, sebacate, fumarate, maleate, butyne-1,4-diacid salt, hexyne-1,6-diacid salt, benzoate, chlorobenzoate, toluate, dinitrobenzoate, hydroxybenzoate, methoxybenzoate, phthalate, sulfonate, methanesulfonate, propanesulfonate, benzene sulfonate, xylene sulfonate, naphthalene-1-sulfonate, naphthalene-2-sulfonate, phenylacetate, phenylpropionate, phenylbutyrate, citrate, lactate, gamma-hydroxybutyrate, glycolate, tartrate and mandelate. A list of other suitable pharmaceutically acceptable salts can be found in Remington: The Science and Practice of Pharmacy, 21st Edition, Lippincott Williams and Wilkins, Philadelphia, Pa., 2006.

Examples of “pharmaceutically acceptable salts” of the compounds disclosed herein also include salts from suitable bases such as alkali metals (e.g., sodium, potassium), alkaline earth metals (e.g., magnesium), ammonium, and N (C₁-C₄ alkyl)⁴⁺. Also included are base addition salts, such as sodium or potassium salts.

Also provided are compounds described herein, or pharmaceutically acceptable salts, isomers, or mixtures thereof, wherein 1 to n hydrogen atoms attached to a carbon atom may be substituted with a deuterium atom or D, wherein n is the number of hydrogen atoms in the molecule. As is known in the art, a deuterium atom is a non-radioactive isotope of a hydrogen atom. Such compounds may increase the resistance to metabolism and may therefore be used to increase the half-life of a compound described herein or a pharmaceutically acceptable salt, isomer, or mixture thereof when administered to mammal. See “Foster, “Deuterium Isotope Effects in Studies of Drug Metabolism”, Trends Pharmacol. Sci., 5(12):524-527 (1984)”; Such compounds are synthesized by methods well known in the art, for example using starting materials in which one or more hydrogen atoms are substituted with deuterium.

Examples of isotopes that may be incorporated into the disclosed compounds also include isotopes of hydrogen, carbon, nitrogen, oxygen, phosphorus, fluorine, chlorine, and iodine, such as ²H, ³H, ¹¹C ¹³C, ¹⁴C ¹³N, ¹⁵N, ¹⁵O, ¹⁷O, ¹⁸O, ³¹P, ³²P, ³⁵S, ¹⁸F, ³⁶Cl, ¹²³I and ¹²⁵I. Substitution with positron emission isotopes such as ¹¹C, ¹⁸F, ¹⁵O and 1³N can be used in positron emission topography (PET) studies to examine substrate receptor occupancy. Isotopically labeled compounds of formula (I-A-1) can generally be prepared by conventional techniques known to those skilled in the art, or by procedures analogous to those described in the Examples below, using the appropriate isotopically labeled reagent in place of the previously used non-labeled reagent.

The compounds of the embodiments disclosed herein, or pharmaceutically acceptable salts thereof, may contain one or more asymmetric centers and thus may produce enantiomeric, diastereomeric, and other stereoisomeric forms, which may be defined in absolute stereochemistry as (R)- or (S)-, or, in the case of amino acids, as (D)- or (L)-. The present disclosure is intended to include all such possible isomers, as well as their racemates and optically pure forms. Optically active (+) and (−), (R) and (S), or (D) and (L)-isomers may be prepared with chiral syntheses or chiral reagent, or resolved by conventional techniques, such as chromatography and fractional crystallization. Conventional techniques for preparing/separating individual enantiomers include chiral synthesis from suitable optically pure precursors, or resolution of racemates (or racemates of salts or derivatives) using, for example, chiral high pressure liquid chromatography (HPLC). When the compounds described herein contain olefinic double bonds or other centers of geometric asymmetry, such compounds are intended to include E and Z geometric isomers unless otherwise specified. Likewise, all isomeric forms are included. When a compound is represented in its chiral form, it is to be understood that this embodiment includes, but is not limited to, specific diastereomeric or enantiomerically enriched forms. If not specified but chirality is present, it is understood that this embodiment is for a particular diastereomeric or enantiomerically enriched form; or a racemic or scalar mixture of such compounds. As used herein, “scalar mixture” refers to a mixture of stereoisomers in a ratio other than 1:1.

As used herein, “stereoisomers” refer to compounds bound by the same atoms but having different three-dimensional structures, which are not interchangeable. The present disclosure contemplates various stereoisomers and mixtures thereof, and includes “enantiomers”, which refer to two stereoisomers whose molecules are non-superimposable mirror images of one another.

As used herein, “tautomer” refers to a proton that is transferred from one atom of one molecule to another atom of the same molecule. In some embodiments, the present disclosure includes tautomers of the compounds.

As used herein, “solvate” refers to the result of the interaction of a solvent and a compound. Also provided are solvates of salts of the compounds described herein. Also provided are hydrates of the compounds described herein.

As used herein, “hydrate” refers to a compound of the invention that is chemically bound to one or more water molecules.

“Prophylaxis” or “prevention” refers to any treatment of a disease or condition that results in the non-progression of the clinical symptoms of the disease or condition. In some embodiments, a compound can be administered to a subject (including a human) at risk of a disease or disorder or a family history.

As used herein, “prodrug” refers to a derivative of a drug that is converted to the parent drug according to some chemical or enzymatic route after administration to the human body. In some embodiments, a prodrug is a biologically active derivative of a drug that is converted to a biologically active parent drug according to certain chemical or enzymatic routes after administration to a human.

As used herein, “treatment” or “treating” or “treat” refers to a method of obtaining a beneficial or desired result. For purposes of the present disclosure, beneficial or desirable results include, but are not limited to, alleviating symptoms and/or alleviating the extent of symptoms and/or preventing worsening of symptoms associated with a disease or condition. In one embodiment, “treatment” or “treating” includes one or more of: a) inhibiting the disease or condition (e.g., reducing one or more symptoms caused by the disease or condition, and/or reducing the degree of the disease or condition); b) slowing or arresting the development of one or more symptoms associated with the disease or condition (e.g., stabilizing the disease or condition, delaying the progression or progression of the disease or condition); and c) alleviating a disease or condition, e.g., causing regression of clinical symptoms, improving disease states, slowing disease progression, improving quality of life, and/or prolonging survival. As used herein, an individual “at risk” refers to an individual at risk of developing a disease in need of treatment. An individual “at risk” may or may not have a detectable disease or condition, and may or may not exhibit a detectable disease prior to the treatment methods described herein. “At risk” means that an individual has one or more so-called risk factors, which are measurable parameters associated with the development of a disease or condition, and which are known in the art to have a higher probability of developing a disease or condition in an individual having one or more of these risk factors than in an individual without these risk factors.

### Compounds

In one aspect, the present disclosure relates to a compound of Formula I, stereoisomers, pharmaceutically acceptable salts and deuterated compounds thereof:

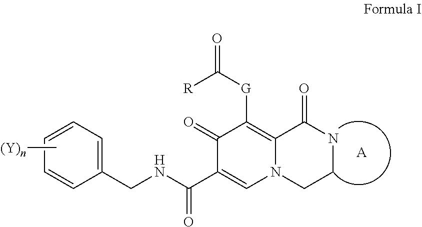

Wherein,
A is selected from the group consisting of

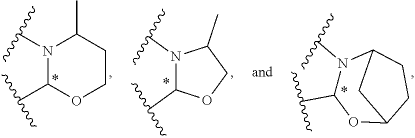

wherein * represents an R or S configuration;
n is 0, 1, 2 or 3;
G is independently selected from oxygen or Gr;
Wherein the Gr is

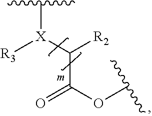

Wherein R₂ and R₃ are each independently selected from the group consisting of hydrogen, hydroxy, cyano, C₁₋₆ alkyl, C₆₋₁₀ aryl, C₃₋₁₀ cycloalkyl, arylheterocyclyl, C₁₋₆ alkyl-C₆₋₁₀ aryl, C₁₋₆ alkyl-arylheterocyclyl and C₁₋₆ alkyl-C₃₋₁₀ cycloalkyl;
Or the R₂ and the R₃ may be combined with an atom to which they are attached to form a 4- to 10-membered heterocyclyl; wherein heterocyclyl may be substituted with 1 to 4 R₄;
The R₄ is independently selected from the group consisting of hydrogen, halogen, C₁₋₆ alkyl and alkoxy;
Or any two R₄ may be combined with an atom to which they are attached to form a C₃₋₁₀ cycloalkyl; wherein cycloalkyl may be substituted with 1 to 4 hydrogen, halogen or C₁₋₆ alkyl;
X is selected from the group consisting of oxygen and nitrogen;
Y is halogen;
m is 0 to 20;
R is independently selected from the group consisting of C₂₋₃₀ saturated or unsaturated hydrocarbyl and aliphatic group, wherein hydrocarbyl and aliphatic group may be optionally substituted with at least one heteroatom selected from the group consisting of N, O and S;
The R may be optionally substituted with a substituent selected from the group consisting of halo, carboxy, sulfonyl, —S═O, —S(═O)₂, aryl, heteroaryl, C₃₋₁₀ saturated or unsaturated carbocycle and C₃₋₁₀ heterocyclyl, wherein the aryl, heteroaryl, carbocycle and heterocyclyl were optionally substituted with H, C₁₋₂₀ alkyl, C₂₋₂₀ alkenyl, C₂₋₂₀ alkynyl, C₁₋₂₀ haloalkyl, C₂₋₂₀ haloalkenyl, C₂₋₂₀ haloalkynyl, C₀₋₂₀ alkyl-C₆₋₁₀ aryl, heterocyclyl, C₀₋₂₀ alkyl-C₃₋₁₀ cycloalkyl, C₀₋₂₀ alkyl-O—C₁₋₆ alkyl and C₀₋₂₀ alkyl-O—C₁₋₆ haloalkyl.

In some embodiments, the G is independently selected from oxygen or Gr;
Wherein the Gr is

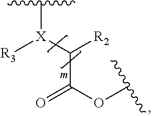

The R₂ and the R₃ are each independently selected from the group consisting of hydrogen, C₁₋₆ alkyl, arylheterocyclyl, C₁₋₆ alkyl-arylheterocyclyl;
Or the R₂ and the R₃ may be combined with an atom to which they are attached to form a 4- to 10-membered heterocyclyl; wherein heterocyclyl may be substituted with 1 to 4 R₄;
The R₄ is independently selected from the group consisting of hydrogen, halogen, C₁₋₆ alkyl and alkoxy;
Or any two R₄ may be combined with an atom to which they are attached to form a C₃₋₁₀ cycloalkyl; wherein cycloalkyl may be substituted with 1 to 4 hydrogen, halogen or C₁₋₆ alkyl;
The R is independently selected from the group consisting of C₂₋₃₀ saturated or unsaturated hydrocarbyl and aliphatic group, wherein hydrocarbyl and aliphatic group may be optionally substituted with at least one heteroatom selected from the group consisting of N, O and S;
The R may be optionally substituted with a substituent selected from the group consisting of halo, aryl, heteroaryl, C₃₋₁₀ saturated or unsaturated carbocycle and C₃₋₁₀ heterocyclyl, wherein the aryl, heteroaryl, C₃₋₁₀ saturated or unsaturated carbocycle and C₃₋₁₀ heterocyclyl were optionally substituted with H, C₁₋₂₀ alkyl, C₂₋₂₀ alkenyl, C₂₋₂₀ alkynyl, C₁₋₂₀ haloalkyl, C₂₋₂₀ haloalkenyl, and C₂₋₂₀ haloalkynyl.

In some embodiments, the G is

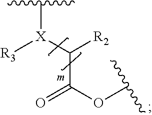

The R is independently selected from the group consisting of C₂₋₃₀ saturated or unsaturated hydrocarby.

Preferably, the R may be optionally substituted with a substituent selected from the group consisting of halo, aryl, heteroaryl, C₃₋₁₀ saturated or unsaturated carbocycle and C₃₋₁₀ heterocyclyl, wherein the aryl, heteroaryl, C₃₋₁₀ saturated or unsaturated carbocycle and C₃₋₁₀ heterocyclyl were optionally substituted with H, C₁₋₂₀ alkyl, C₂₋₂₀ alkenyl, C₂₋₂₀ alkynyl, C₁₋₂₀ haloalkyl, C₂₋₂₀ haloalkenyl, and C₂₋₂₀ haloalkynyl.

In some embodiments, the G is oxygen;
Preferably, the R is a heterocycloalkyl having a heteroatom substituted with C₁₀₋₂₀ alkyl-C(O)—; preferably, the R is a heterocycloalkyl having a heteroatom substituted with C₁₅₋₁₈ alkyl-C(O)—; preferably, the R is a heterocycloalkyl having a heteroatom substituted with C₁₅ linear alkyl-C(O)—; wherein, the heteroatom in the heterocycloalkyl being an N atom; preferably, the heterocycloalkyl is a four-membered heterocycloalkyl or a five-membered heterocycloalkyl, and carbon atoms in the heterocycloalkyl are preferably substituted with H or halogen atoms; further, preferably, the R is selected from any one or more of

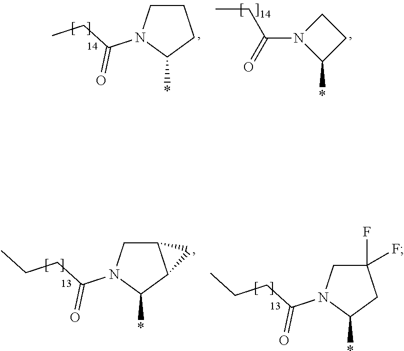

Or the R is a saturated C₁₀₋₂₀ alkyl, preferably a saturated C₁₅₋₁₈ alkyl, wherein one or more methylene groups are replaced by —O—; further, preferably, the R is

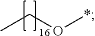

Or the R is a C₁₀₋₂₀ linear alkyl with an end group substituted by an alkynyl or a carboxyl; preferably, the R is a C₁₁₋₁₆ linear alkyl with an end group substituted by an alkynyl or a carboxyl; further, preferably, the R is selected from

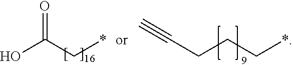

In some embodiments, the G is

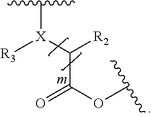

The R is independently selected from the group consisting of unsaturated hydrocarby and C₀₋₁₈ alky-heteroaryl-C₀₋₁₈alky.

Preferably, the R may be optionally substituted with a substituent selected from the group consisting of halo, aryl, heteroaryl, C₃₋₁₀ saturated or unsaturated carbocycle and C₃₋₁₀ heterocyclyl.

In some embodiments, the R is independently selected from the group consisting of —C₁₋₂₀alkyl-C≡C—C₀₋₂₀alkyl; preferably, the R is independently selected from the group consisting of —C₅₋₁₀alkyl-C≡C—C₅₋₁₀alkyl; preferably, the R is independently selected from the group consisting of —C₆₋₇ alkyl-C≡C—C₅₋₉ alkyl; wherein the alkyl was optionally substituted with halo.

In some embodiments, the R is independently selected from the group consisting of —C₀₋₁₈alkyl-heteroaryl-C₀₋₁₈alkyl; preferably, the R is independently selected from the group consisting of —C₅₋₁₂ alkyl-heteroaryl-C₃₋₁₀ alkyl; preferably, the R is independently selected from the group consisting of —C₇₋₁₁ alkyl-heteroaryl-C₃₋₈alkyl; wherein the alkyl was optionally substituted with halo.

In some embodiments, the heteroaryl is

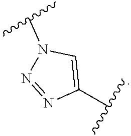 [SMILES: *c1cn(*)nn1]

In some embodiments, the compound of Formula I is selected from one of the following structures:

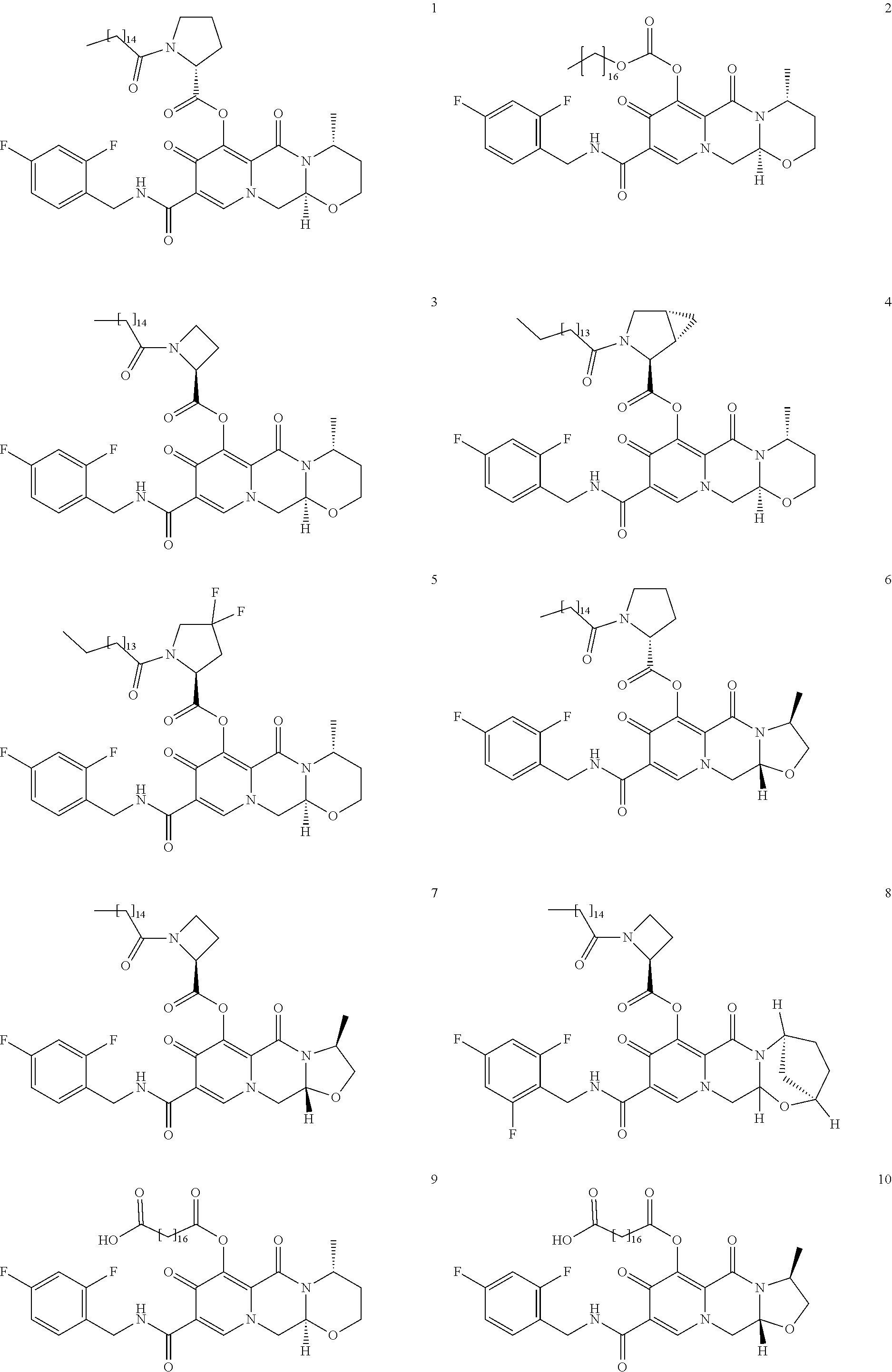

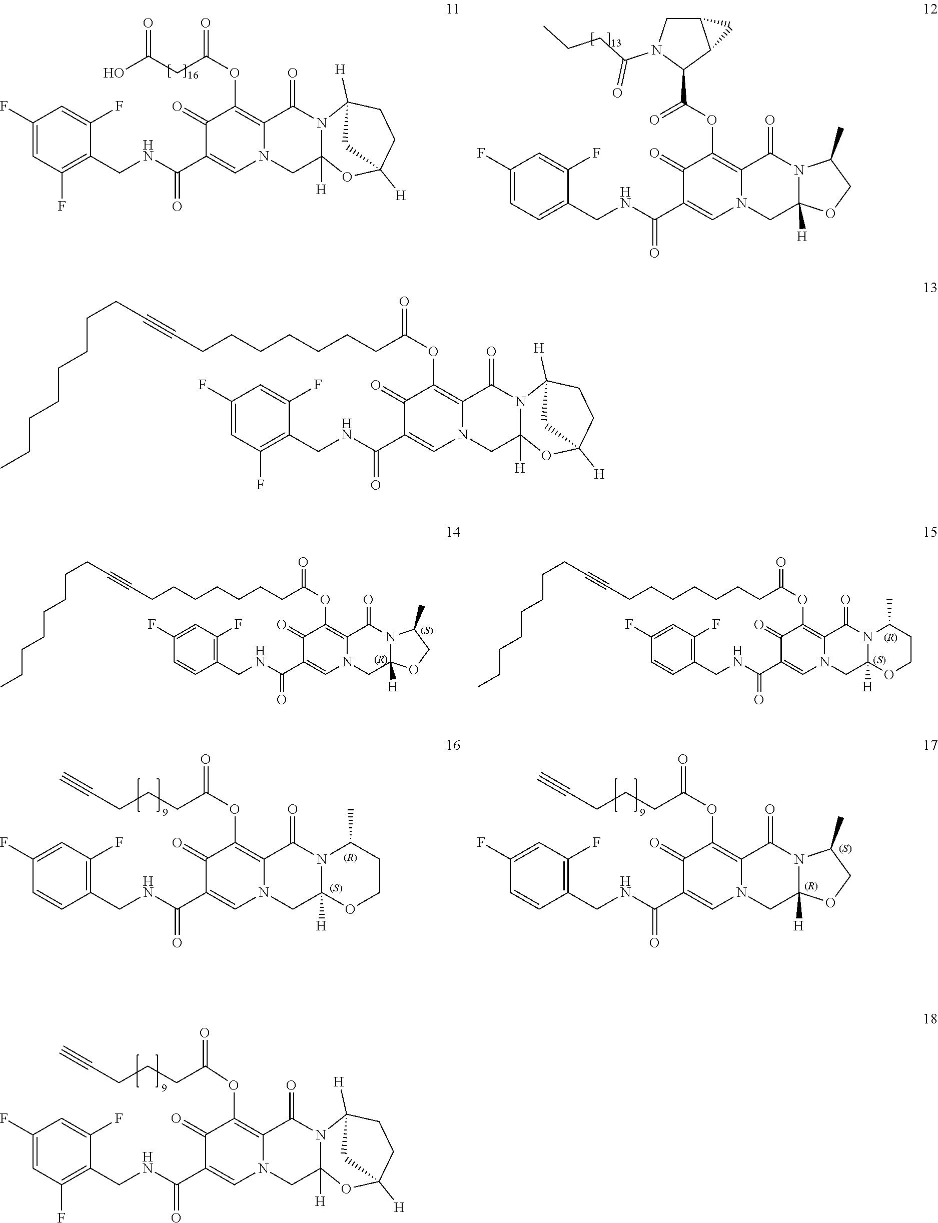

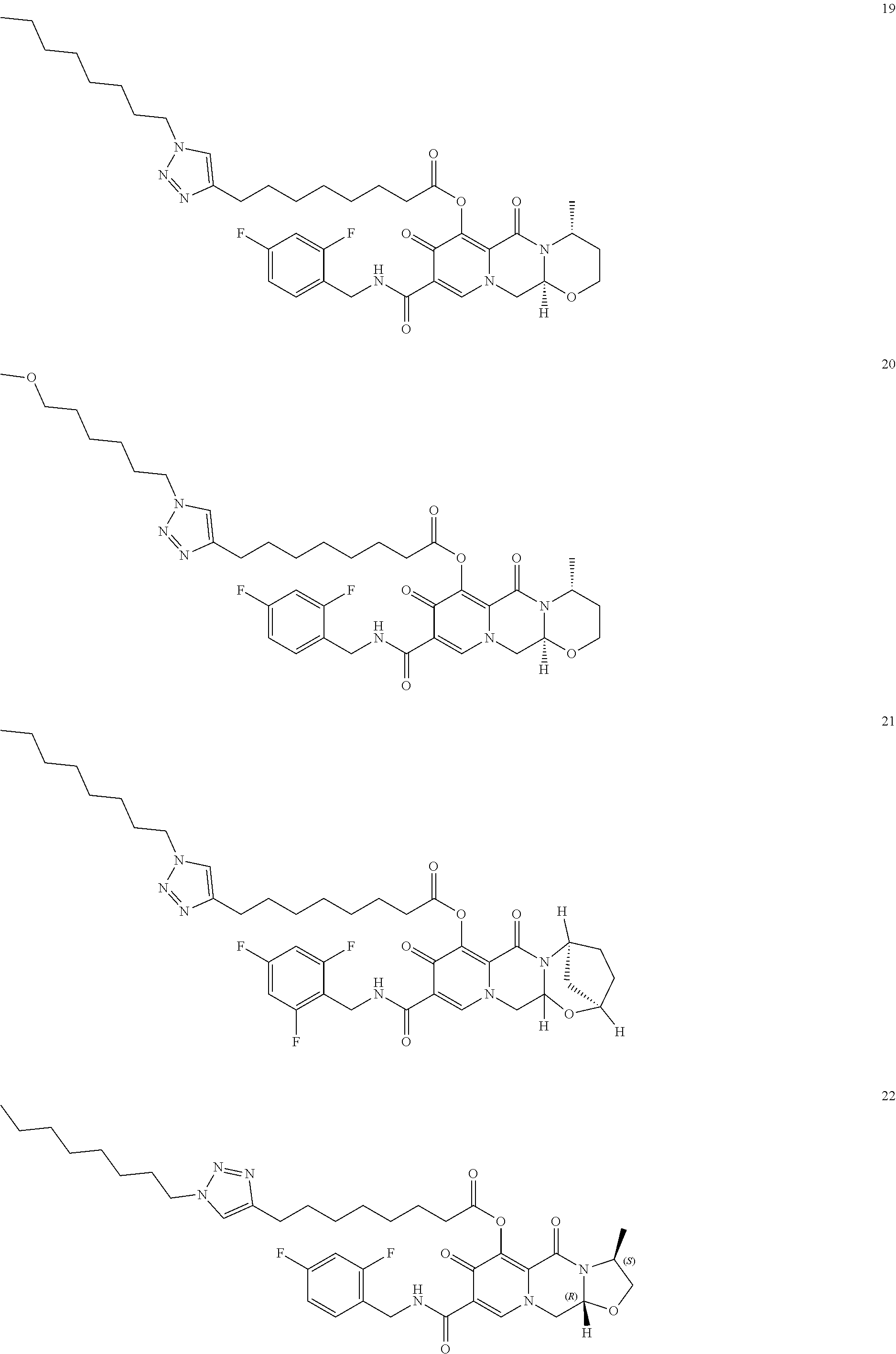

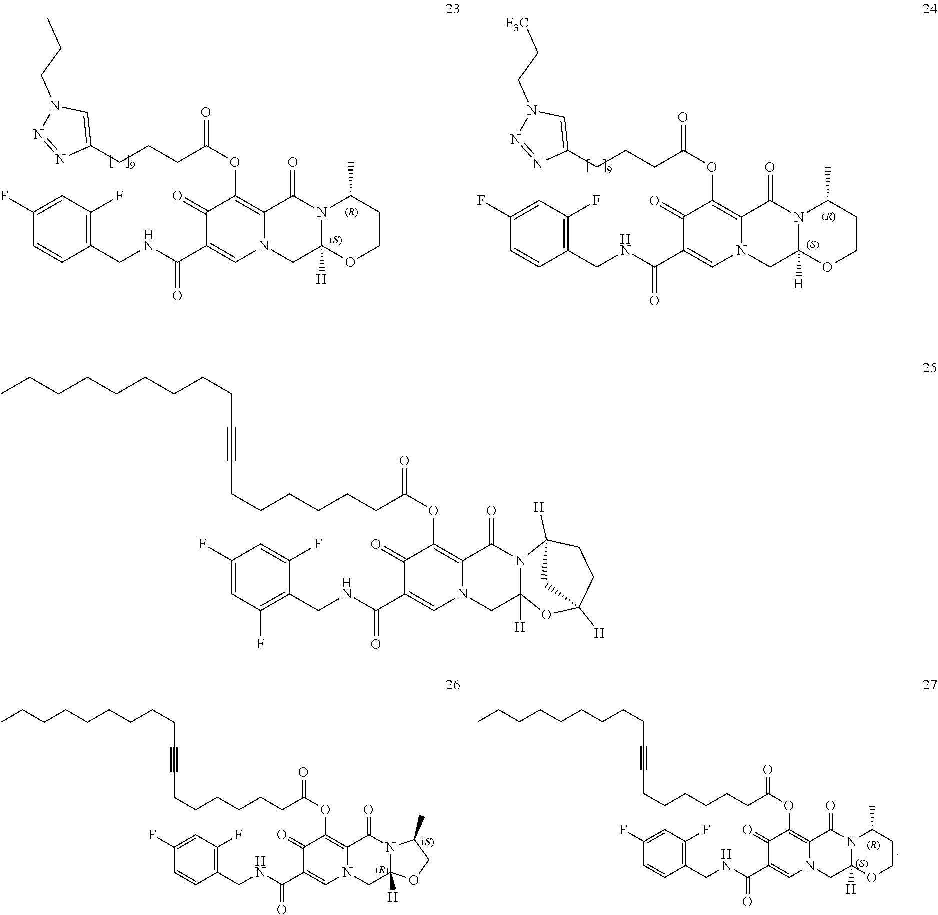

### Method for Treatment

In one aspect, the present disclosure relates to a method for treating, inhibiting and/or preventing a disease or disorder in a subject in need thereof, comprising administering an effective amount of the compound of Formula I, a stereoisomer, a pharmaceutically acceptable salt or a deuterated compound thereof of the present disclosure to the subject.

In another aspect, the present application relates to a composition comprising a compound of formula I, stereoisomers, pharmaceutically acceptable salts or deuterated compounds thereof of the present application.

In some embodiments, the disease or disorder of the present disclosure comprises, but is not limited to, a viral infection.

In some embodiments, the viral infection of present disclosure comprises, but is not limited to, HIV infection.

In some embodiments, the compound of Formula I, a stereoisomer, a pharmaceutically acceptable salt or a deuterated compound thereof of the present disclosure is administered to the subject monthly.

In some embodiment, the compound of Formula I, a stereoisomer, a pharmaceutically acceptable salt or a deuterated compound thereof of the present disclosure is administered to the subject once every 2 months.

In some embodiments, the compound of Formula I, a stereoisomer, a pharmaceutically acceptable salt or a deuterated compound thereof of the present disclosure is administered to the subject once every 6 months.

In some embodiments, the compound of Formula I, a stereoisomer, a pharmaceutically acceptable salt or a deuterated compound thereof of the present disclosure is administered to the subject once every 12 months.

In some embodiments, the method comprises administering at least one further anti-HIV agent.

### EXAMPLES

The present disclosure is further illustrated below in connection with specific examples. It is to be understood that these examples are merely illustrative of the present disclosure and are not intended to limit the scope of the disclosure. Experimental methods not specified in the following examples generally follow conventional conditions or conditions suggested by the manufacturer.

Unless defined otherwise, all professional and scientific terms used herein have the same meaning as is familiar to those skilled in the art. In addition, any methods and materials similar or equivalent to those described herein can be applied to the disclosed methods. The preferred embodiments and materials described herein are exemplary only.

The above-mentioned features mentioned in the present disclosure, or the features mentioned in the embodiments, may be combined in any combination. All features disclosed in this patent specification may be used in any composition form, and each feature disclosed in the specification may be replaced by any alternative feature that may provide the same, equal or similar purpose. Thus, unless specifically stated otherwise, the disclosed features are merely general examples of equivalents or similar features.

### Preparation

The compounds of the present disclosure may be prepared using the methods disclosed herein and conventional modifications thereof, which will be apparent in view of the methods disclosed herein and methods well-known in the art. In addition to the teachings herein, conventional and well-known preparation methods may also be used. The preparation of a typical compound of Formula I, a stereoisomer, a pharmaceutically acceptable salt and a deuterated compound thereof, e.g., a compound having one or more of the structures described in Formula I may be accomplished as described in the following examples.

### Preparation Method

Typical embodiments of the compounds of the present disclosure may be prepared using the general reaction schemes and/or examples described below. In view of the description herein, it will be apparent that the general scheme can be modified by replacing the starting materials with other materials of similar construction, resulting in correspondingly different products. The following preparation description provides many examples of how the starting materials may be varied to provide the corresponding products. Starting materials are generally obtained from commercial sources, or are prepared using published methods of preparing compounds, which are embodiments of the present disclosure. Checking the structure of the compound to be prepared will provide the identity of each substituent. In view of the examples herein, the identity of the final products will generally make the identity of the necessary starting materials apparent by a simple checking process. The group labels (e.g., R¹, R²) used in the reaction schemes herein are for illustrative purposes only and, unless otherwise indicated, do not necessarily correspond in name or function to the labels used elsewhere to describe the compounds of Formula I or aspects or fragments thereof.

### Parameters of Preparation Reaction

The compounds of the present disclosure can be prepared from readily available starting materials using, for example, the following general methods and procedures. It will be appreciated where typical or preferred process conditions are given (i.e., reaction temperature, time, molar ratio of reactants, solvent, pressure, etc.), other process conditions may also be used unless otherwise stated. The optimum reaction conditions may vary with the particular reactants or solvents used, but such conditions may be determined by one skilled in the art by routine optimization procedures.

Furthermore, conventional protective groups may be necessary to one skilled in the art to prevent undesirable reactions of certain functional groups. Protective groups suitable for various functional groups and suitable conditions for protecting and deprotecting particular functional groups are well-known in the art. For example, a number of protective groups are described in T. W. Greene and G. M. Wuts (1999) Protective Groups in Organic Synthesis, Third Edition, Wiley, New York, and references cited therein.

Moreover, the compounds of the present disclosure may contain one or more chiral centers. Thus, if desired, such compounds may be prepared or separated as pure stereoisomers, i.e., as individual enantiomers or diastereomers or as stereoisomer-enriched mixtures. All such stereoisomers (and enriched mixtures) are included within the scope of the present disclosure unless otherwise indicated. Pure stereoisomers (or enriched mixtures) may be prepared using, for example, optically active starting materials or stereoselective reagents well-known in the art. In addition, racemic mixtures of such compounds can be separated by, for example, chiral column chromatography, chiral decomposition agents, and the like.

The starting materials for the following reactions are generally known compounds or may be prepared by known procedures or obvious modifications thereof. For example, many starting materials are available from commercial suppliers such as Aldrich Chemical Co. (Milwaukee, Wisconsin, USA). Other modifications may be made by the procedures described in the standard references, or obvious modifications thereof, such as Fieser's Reagents for Organic Synthesis, Vol. 1-15 (John Wiley, and Sons, 1991); Rodd's Chemistry of Carbon Compounds, Vol. 1-5, and Supplement Version (Elsevier Scientific Press, 1989); Organic Reactions, Vol. 1-40 (John Wiley, and Sons, 1991), March's Advanced Organic Chemistry (John Wiley, and Sons, 5th Edition, 2001), and Larock's Comprehensive Organic Transformations (VCH Publishing Company, 1989).

The term “solvent”, “inert organic solvent” or “inert solvent” refers to inert solvents under the reaction conditions associated therewith (including, for example, benzene, toluene, acetonitrile, tetrahydrofuran (“THF”), N,N-dimethylformamide (“DMF”), chloroform, dichloromethane, diethyl ether, methanol, pyridine, and the like). Unless specified to the contrary, the solvent used in the reaction of the present disclosure is an inert organic solvent and the reaction is carried out under an inert gas, preferably nitrogen.

The term “q.s.” refers to the addition of an amount sufficient to effect the function, e.g., to bring the solution to the desired volume (i.e., 100%).

The compounds provided herein can be prepared according to the general protocols provided below. In the following schemes, it is to be understood that each compound shown therein may have a protective group present as desired in any step. Standard protective groups are within the scope of one skilled in the art.

### Example 1

### Preparation Schemes of Compounds of Formula I

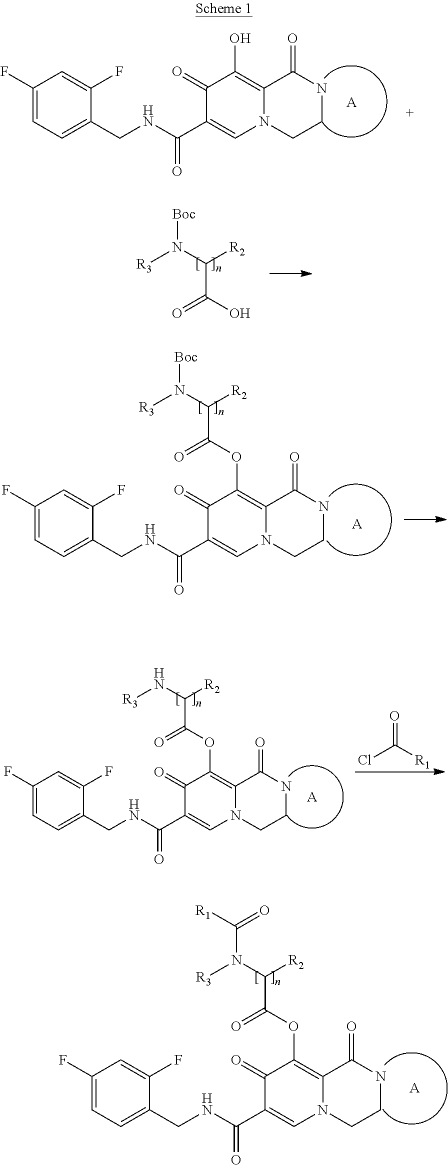

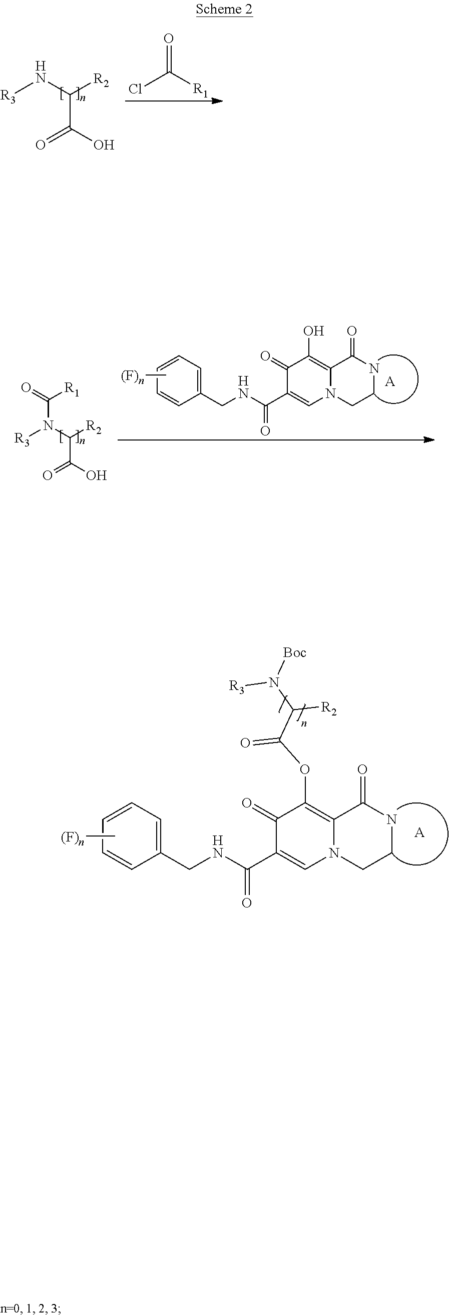

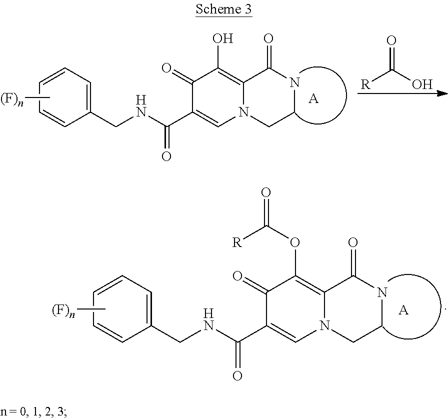

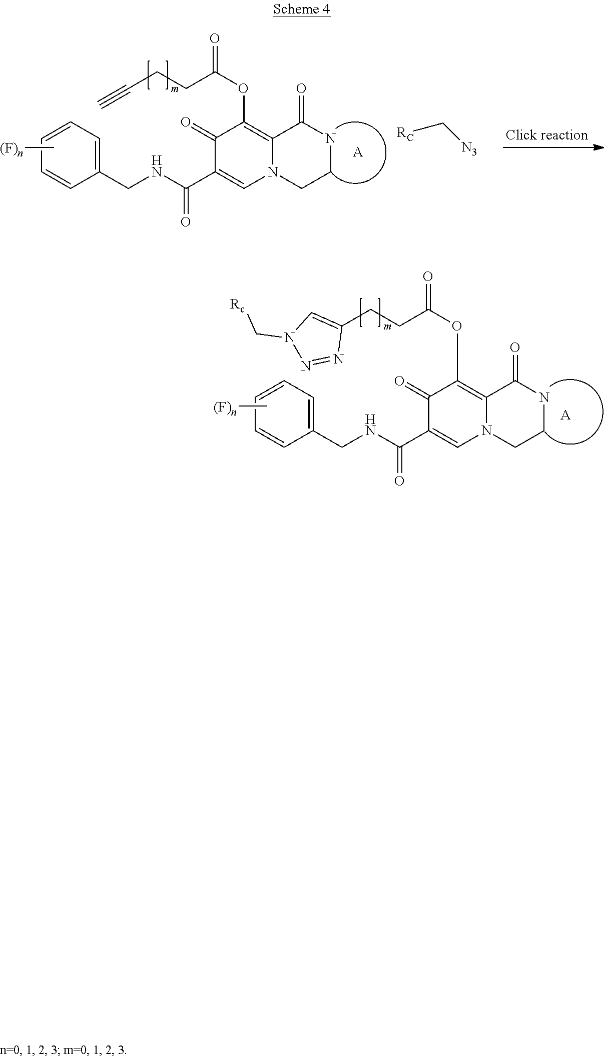

### Example 2

### Preparation of Compound 1: (4R,12aS)-9-((2,4-difluorobenzyl)carbamoyl)-4-methyl-6,8-dioxo-3,4,6,8,12,12a-hexahydro-2H-pyrido[1′,2′:4,5]pyrazino[2,1-b][1,3]oxazin-7-yl (S)-1-palmitoylpyrrolidine-3-carboxylate (1)

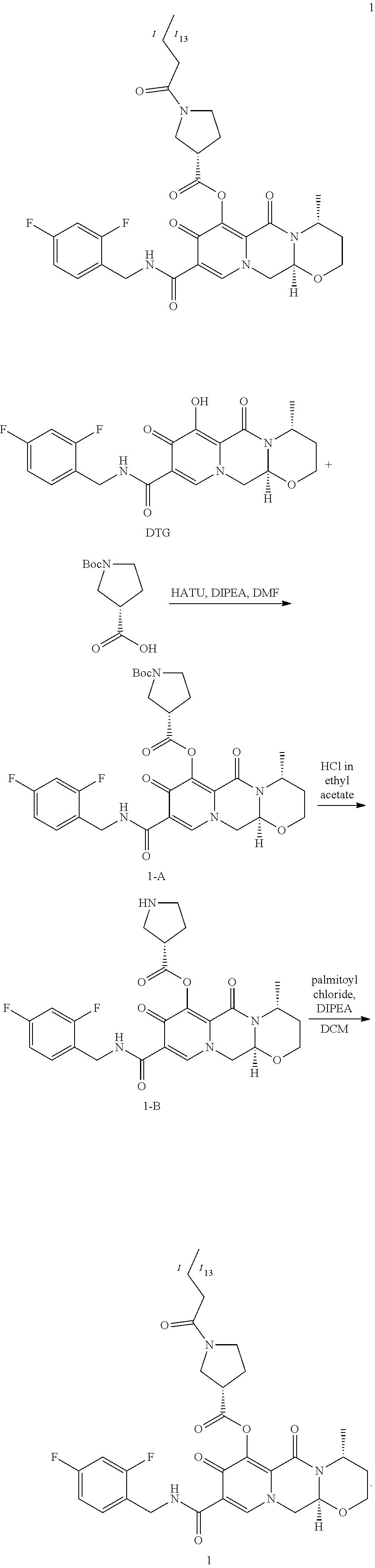

To DMF (100 ml) were added DTG (10 g, 23.8 mmol), (S)-1-(tert-butoxycarbonyl)pyrrolidine-3-carboxylic acid (6.16 g, 28.6 mmol), HATU (13.6 g, 35.7 mmol) and DIPEA (6.15 g, 47.6 mmol). The resultant was stirred at room temperature for 2 hours. Water (500 ml) was added to the reaction solution to precipitate a solid. The solid was filtered to obtain intermediate 1-A (12.3 g, yield: 83.7%).

To ethyl hydrogen chloride acetate solution (2M, 100 ml) was added compound 1-A (12.3 g, 19.95 mmol). The resultant was stirred at room temperature overnight. A solid was precipitated and filtered to give the compound 1-B (9.7 g, yield: 94.2%).

To dichloromethane (50 ml) were added compound 1-B (8.0 g, 15.5 mmol) and DIPEA (5.0 g, 38.7 mmol). Palmitoyl chloride (5.11 g, 18.6 mmol) was dissolved in dichloromethane (25 ml). The resultant mixture was added dropwise to a reaction at 0° C. The reaction was stirred for 3 hours. Water (100 ml) was added and the organic phase was extracted. The organic phase was concentrated under reduced pressure and purified by column chromatography (dichloromethane/methanol 20:1) to give the title compound 1 (6.4 g, yield: 54.7%).

### Preparation of Compound 3: (4R,12aS)-9-((2,4-difluorobenzyl)carbamoyl)-4-methyl-6,8-dioxo-3,4,6,8,12,12a-hexahydro-2H-p-pyridine[1′,2′:4,5]pyrazino[2,1-b][1,3]oxazin-7-yl (S)-1-palmitoylazetidine-2-carboxylate (3)

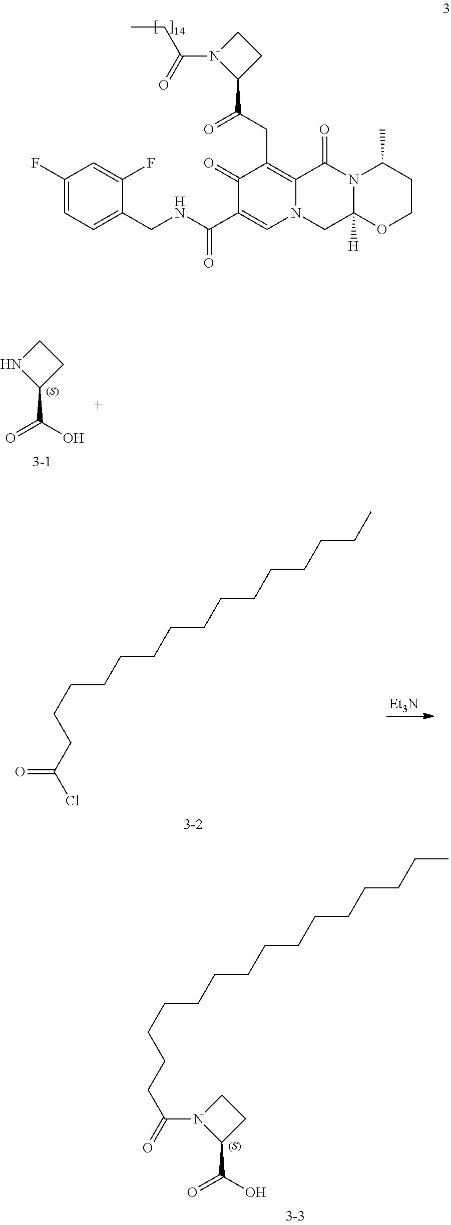

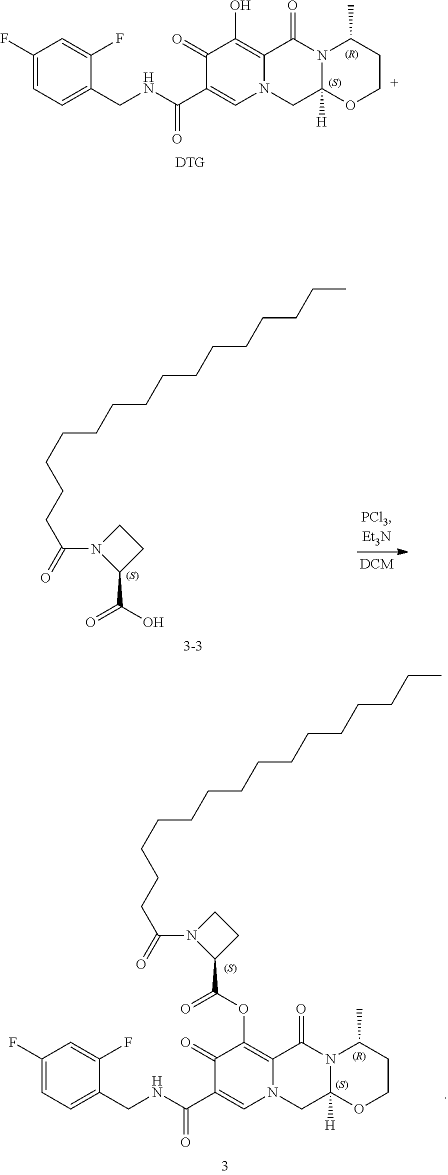

Compound 3-1 (5.00 g, 49.45 mmol) and triethylamine (7.51 g, 74.18 mmol) were dissolved in dichloromethane (50 ml) to give a mixture. Palmitoyl chloride (16.31 g, 59.34 mmol) was added dropwise to the mixture in an ice bath to react for 1 hour. After completion of the reaction, water (50 ml) was added. The organic phase was extracted and separated. The organic phase was washed twice with brine and concentrated to dryness under reduced pressure to obtain a white solid (9.3 g, yield: 55.4%).

DTG (1.0 g, 2.38 mmol), compound 3-3 (1.62 g, 4.77 mmol) and triethylamine (0.97 g, 9.54 mmol) were dissolved in dichloromethane (10 ml) to give a mixture. Phosphorus oxychloride (0.49 g, 3.58 mmol) was added dropwise to the mixture in an ice bath and the reaction was subject to heat preservation for 1 hour. The reaction was quenched by addition of water (0.5 ml). After concentration to dryness under reduced pressure, a solid was subject to column chromatography to give the title compound (550 mg, yield: 31.1%).

### Preparation of Compound 19: (4R,12aS)-9-((2,4-difluorobenzyl)carbamoyl)-4-methyl-6,8-dioxo-3,4,6,8,12,12a-hexahydro-2H-pyrido[1′,2′:4,5]pyrazino[2,1-b][1,3]oxazin-7-yl 8-(1-octyl-1H-1,2,3-triazol-4-yl)octanoate (19)

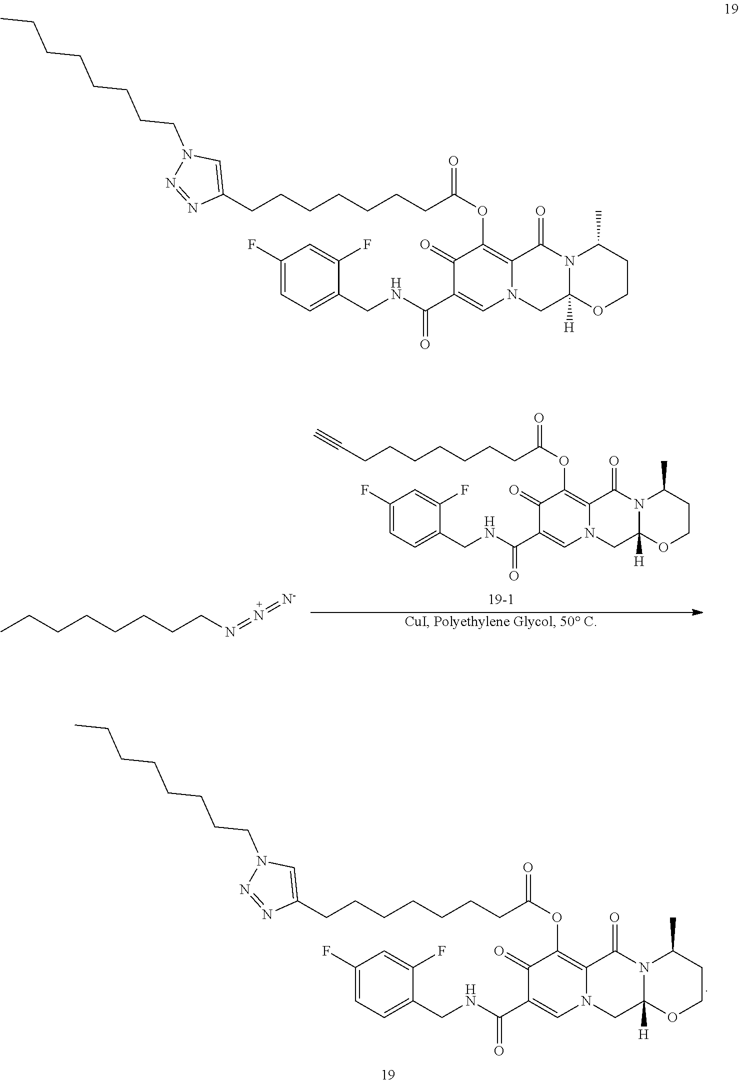

To a solution of 1-azidooctane (0.4 g, 2.58 mmol) and 19-1 (1.62 g, 2.84 mmol) in PEG, poly(ethylene glycol) (10 mL) was added iodocopper (0.098 g, 0.52 mmol), then the reaction mixture was stirred at 50° C. for 3 h. Then the mixture was added Brine (20 mL) and stirred for 10 min. The aqueous layer was extracted with EtOAc (30 mL) twice. The combined organic layers were washed with Brine (30 mL) for 3 times and dried over Na₂SO₄. The organic layer was filtered and concentrated under reduced pressure to get residue. The crude product was purified by silica gel chromatography eluted with PE:EtOAc=2:1 to give product 19: (4R,12aS)-9-((2,4-difluorobenzyl)carbamoyl)-4-methyl-6,8-dioxo-3,4,6,8,12,12a-hexahydro-2H-pyrido[1′,2′:4,5]pyrazino[2,1-b][1,3]oxazin-7-yl-8-(1-octyl-1H-1,2,3-triazol-4-yl)octanoate

Following the Preparation of Compound 19, compounds 20, 21, 22, 23, and 24 can be obtained by the Click-Reaction.

According to the preparation methods described herein, the following compounds are prepared using the appropriate starting materials and the appropriate protective group chemicals as desired.

| No. | Structure | ¹HNMR (400M, CDCl₃) |
|---|---|---|
| 1 | 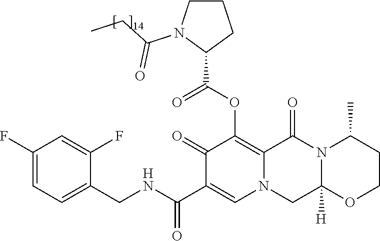 | ¹HNMR (400M , CDCl₃) δ 10.08 (t, J = 8 Hz, 1H), 8.42 (s, 1H), 7.34-7.28 (m, 1H), 6.82-6.74 (m, 2H), 5.25-5.20 (m, 1H), 5.08- 5.02 (m, 1H), 4.68-4.52 (m, 2H), 4.63-4.57 (m, 1H), 4.32-4.24 (m, 1H), 4.19- 3.97 (m, 7H), 2.85-2.71 (m, 2H), 2.40-2.03 (m, 6H), 1.89-1.80 (m, 2H), 1.63- 1.55 (m, 3H), 1.53- 1.49 (m,1H), 1.40-1.35 (m, 1H), 1.32-1.16 (m, 22H), 0.93-0.84 (m, 3H) |
| 2 | 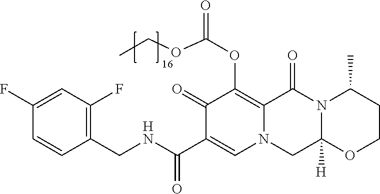 | ¹HNMR (400M, d-DMSO) δ 10.15 (t, J = 8 Hz, 1H), 8.65 (s, 1H), 7.38-7.32 (m, 1H), 7.21-7.15 (m, 1H), 7.03-6.98 (m, 1H), 5.37- 5.35 (m, 1H), 4.69-4.66 (m, 1H), 4.60-4.56 (m, 1H), 4.59 (d, J = 4 Hz, 2H), 4.41- 4.36 (m, 1H), 4.02 (t, J = 8 Hz, 2H), 3.98-3.91 (m, 1H), 3.84-3.79 (m, 1H), 1.89-1.87 (m, 1H), 1.63- |
| | | 1.55 (m, 2H), 1.47-1.43 (m, |
| | | 1H), 1.35-1.30 (m, 2H), |
| | | 1.26-1.15 (m, 29H), 0.80 (t, |
| | | J = 8 Hz, 3H) |
| 3 | 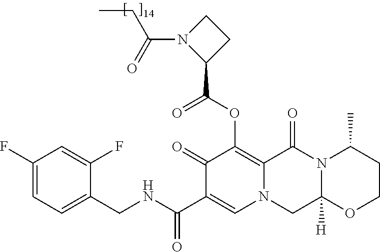 | δ 10.08 (t, J = 8 Hz, 1H), 8.42 (s, 1H), 7.34-7.28 (m, 1H), 6.82-6.74 (m, 2H), 5.25-5.20 (m, 1H), 5.18- 5.12 (m, 1H), 4.98-4.88 (m, 1H), 4.63-4.57 (m, 2H), 4.32-4.24 (m, 1H), 4.19- 3.97 (m, 7H), 2.85-2.71 (m, 2H), 2.40-2.03 (m, 4H), 1.63-1.55 (m, 3H), 1.53- 1.49 (m, 1H), 1.40-1.35 (m, 1H), 1.32-1.16 (m, 22H), 0.93-0.84 (m, 3H) |
| 4 | 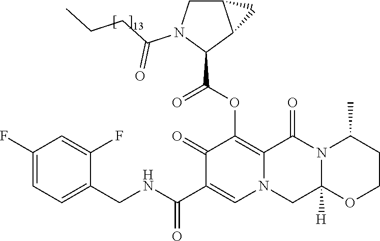 | ¹HNMR (400M, CDCl₃) δ 10.08 (t, J = 8 Hz, 1H), 8.42 (s, 1H), 7.34-7.28 (m, 1H), 6.82-6.74 (m, 2H), 5.29-5.19 (m, 1H), 4.94- 4.89 (m, 1H), 4.66-4.52 (m, 3H), 4.34-4.27 (m, 1H), 4.18-4.10 (m, 1H), 3.99- 3.94 (m, 2H), 3.78-3.73 (m, 1H), 3.69-3.63 (m, 1H), 2.38-2.30 (m, 1H), 2.25- 2.11 (m, 3H), 1.63-1.55 (m, 3H), 1.53-1.48 (m, 2H), 1.35-1.21 (m, 26H), 0.98- 0.97 (m, 3H), 0.76-0.70 (m, 2H). |
| 5 | 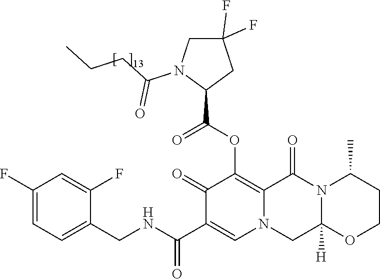 | ¹HNMR (400M, CDCl₃) δ 10.17 (t, J = 8 Hz, 1H), 8.44 (s, 1H), 7.34-7.28 (m, 1H), 6.84-6.74 (m, 2H), 5.23-5.19 (m, 1H), 5.00- 4.84 (m, 2H), 4.69-4.53 (m, 3H), 4.34-4.25 (m, 1H), 4.19-4.10 (m, 2H), 3.99- 3.94 (m, 4H), 3.88-3.79 (m, 1H), 3.31-3.15 (m, 1H) 3.02-2.82 (m, 1H), 2.51-2.35 (m, 1H), 2.28- 2.24 (m, 1H), 2.20- 2.09 (m, 1H), 1.68-1.60 (m, 3H), 1.52-1.46 (m, 2H), 1.40-1.20 (39H), 0.89- 0.82 (m, 3H). |
| 6 | 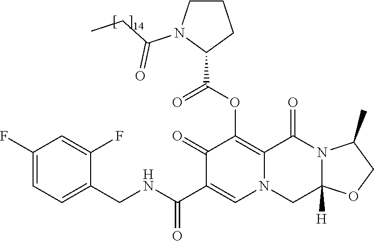 | ¹HNMR (400M, CDCl₃) δ 10.18 (t, J = 8 Hz, 1H), 7.93 (s, 1H), 7.34-7.28 (m, 1H), 7.20-7.18 (m, 1H), 7.03-7.01 (m, 1H), 5.18-5.12 (m, 1H), 5.08- 5.02 (m, 1H), 4.68-4.52 (m, 2H), 4.48-4.43 (m, 1H), 4.32- 4.24 (m, 3H), 4.19-3.97 (m, 3H), 3.57-3.72 (m, 1H), 2.85-2.71 (m, 2H), 2.18- 2.13 (m, 1H), 1.63-1.55 (m, 3H), 1.53-1.49 (m, 2H), 1.32-1.16 (m, 26H), 0.93- 0.84 (m, 3H) |
| 7 | 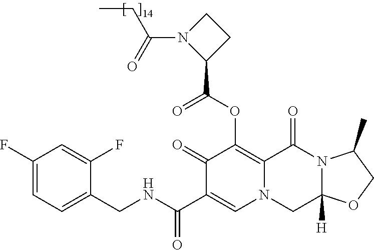 | ¹HNMR (400M, CDCl₃) δ 10.18 (t, J = 8 Hz, 1H), 7.93 (s, 1H), 7.34-7.28 (m, 1H), 7.20-7.18 (m, 1H), 7.03-7.01 (m, 1H), 5.25-5.20 (m, 1H), 4.98- 4.88 (m, 1H), 4.63-4.57(m, 2H), 4.48-4.43 (m, 1H), 4.32- 4.24 (m, 3H), 4.19-3.97 (m, 3H), 3.57-3.72 (m, 1H), 2.18-2.13 (m, 1H), 1.63- 1.55 (m, 3H), 1.53- 1.49 (m, 2H), 1.32-1.16 (m, 26H), 0.93-0.84 (m, 3H) |
| 8 | 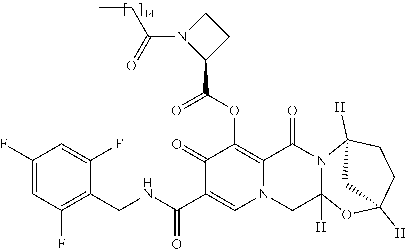 | ¹HNMR (400M, CDCl₃) δ 10.23 (t, J = 8 Hz, 1H), 8.08 (s, 1H), 6.98-6.88 (m, 2H), 5.25-5.20 (m, 1H), 5.18- 5.12 (m, 1H), 4.98-4.88 (m, 1H), 4.63-4.57 (m, 2H), 4.32-4.24 (m, 1H), 4.19- 3.97 (m, 7H), 2.85-2.71 (m, 2H), 2.40-2.03 (m, 4H), 1.63-1.55 (m, 3H), 1.53- 1.49 (m, 1H), 1.40-1.35 (m, 3H), 1.32-1.16 (m, 26H), 0.93-0.84 (m, 3H) |
| 9 | 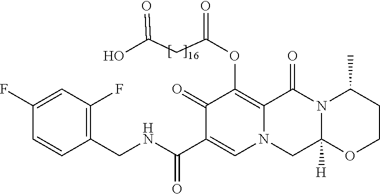 | ¹HNMR (400M, d-DMSO) δ 11.95 (s, 1H), 10.14 (t, J = 8 Hz, 1H), 8.64 (s, 1H), 7.38-7.32 (m, 1H), 7.22- 7.16 (m, 1H), 7.04-6.99 (m, 1H), 5.37-5.34 (m, 1H), 4.71-4.64 (m, 1H), 4.60- 4.56 (m, 1H), 4.50-4.49 (m, 2H), 4.41-4.36 (m, 1H), 4.02-3.91 (m, 2H), 3.84- 3.78 (m, 1H), 2.54-2.50 (m, 2H), 2.16-2.12 (m, 3H), |
| | | 1.96-1.95 (m, 2H), 1.92- |
| | | 1.87 (m, 1H), 1.62-1.55 (m, |
| | | 3H), 1.69-1.39 (m, 6H), |
| | | 1.37-1.31 (m, 4H), 1.23- |
| | | 1.16 (m, 15H). |
| 10 | 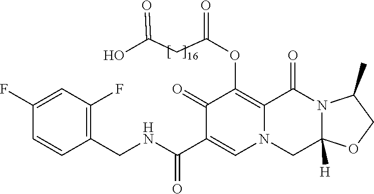 | ¹HNMR (400M, d-DMSO) δ 11.90 (s, 1H), 10.18 (t, J = 8 Hz, 1H), 7.93 (s, 1H), 7.34-7.28 (m, 1H), 7.20- 7.18 (m, 1H),7.03-7.01 (m, 1H), 5.35-5.33 (m, 1H), 4.70-4.64 (m, 1H), 4.60- 4.56 (m, 1H), 4.50-4.47 (m, 2H), 4.41-4.36 (m, 1H), 4.02-3.91 (m, 2H), 3.84- 3.78 (m, 1H), 2.54-2.50 (m, 2H), 2.16-2.12 (m, 3H), |
| | | 1.96-1.95 (m, 2H), 1.92- |
| | | 1.87 (m, 1H), 1.62-1.55 (m, |
| | | 3H), 1.53-1.39 (m, 3H), |
| | | 1.37-1.31 (m, 4H), 1.23- |
| | | 1.16 (m, 18H). |
| 11 | 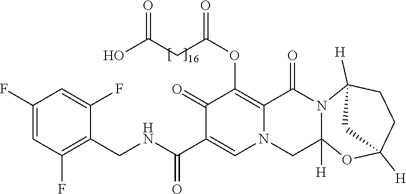 | ¹HNMR (400M, d-DMSO) δ 11.95 (s, 1H), 10.23 (t, J = 8 Hz, 1H), 8.08 (s, 1H), 6.98-6.88 (m, 2H), 7.04- 6.99 (m, 1H), 5.37-5.34 (m, 1H), 4.71-4.64 (m, 1H), 4.60-4.56 (m, 1H), 4.52- 4.49 (m, 1H), 4.41-4.36 (m, 1H), 4.02-3.91 (m, 1H), 3.84-3.78 (m, 1H), 2.54- 2.50 (m, 2H), 2.16-2.12 (m, 3H), 1.96-1.95 (m, 2H), |
| | | 1.92-1.87 (m, 1H), 1.62- |
| | | 1.55 (m, 3H), 1.55-1.39 (m, |
| | | 4H), 1.37-1.31 (m, |
| | | 4H), 1.23-1.16 (m, 18H). |
| 12 | 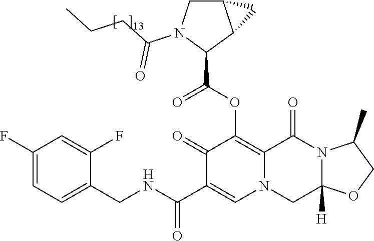 | ¹HNMR (400M, d-DMSO) δ 10.18 (t, J = 8 Hz, 1H), 7.93 (s, 1H), 7.34-7.28 (m, 1H), 7.20-7.18 (m, 1H), 7.03-7.01 (m, 1H), 5.29-5.19 (m, 1H), 4.94- 4.89 (m, 1H), 4.66-4.52 (m, 3H), 4.34-4.27 (m, 1H), 4.18-4.10 (m, 1H), 3.99- 3.94 (m, 2H), 3.78-3.73 (m, 1H), 3.69-3.63 (m, 1H), 2.38-2.30 (m, 1H), 2.25- 2.11 (m, 3H), 1.63-1.55 (m, 3H), 1.53-1.48 (m, 2H), 1.35-1.21 (m, 27H), 0.98- |
| | | 0.97 (m, 3H), 0.76-0.70 (m, |
| | | 2H). |
| 13 | 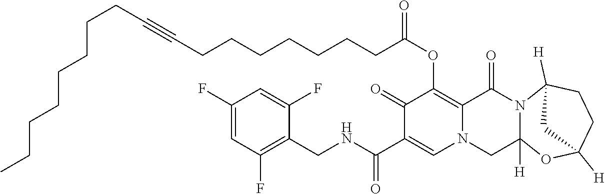 [SMILES: CCCCCCCCC#CCCCCCCCC(=O)Oc1c2n(cc(C(=O)NCc3c(F)cc(F)cc3F)c1=O)CC1O[C@@H]3CC[C@@H](C3)N1C2=O] | ¹HNMR (400M, d-DMSO) δ10.23 (t, J = 8 Hz, 1H), 8.08 (s, 1H), 6.98-6.88 (m, 2H), 5.37-5.35 (m, 1H), 4.70- 4.65 (m, 1H), 4.61-4.56 (m, 1H), 4.50 (d, J = 8 Hz, 1H), 4.41-4.36 (m, 1H), 3.97- 3.91 (m, 1H), 3.84-3.80 (m, 1H), 2.54-2.51 (m, 2H), 2.09-2.04 (m, 3H), 1.95- 1.84 (m, 1H), 1.63-1.56 (m, 1H), 1.48-1.44 (m, 1H), |
| | | 1.29-1.19 (m, 23H), 0.84- |
| | | 0.79 (m, 3H). |
| 14 | 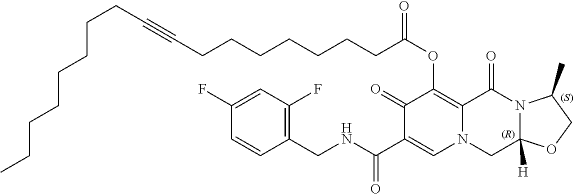 | ¹HNMR (400M d-DMSO) δ10.18 (t, J = 8 Hz, 1H), 7.93 (s, 1H), 7.34-7.28 (m, 1H), 7.20-7.18 (m, 1H), 7.03-7.01 (m, 1H), 5.37-5.35 (m, 1H), 4.70- 4.65 (m, 1H), 4.61-4.56 (m, 1H), 4.50 (d, J = 8 Hz, 1H), 4.41-4.36 (m, 1H), 3.97- 3.91 (m, 1H), 3.84-3.80 (m, 1H), 2.54-2.51 (m, 2H), 2.09-2.04 (m, 4H), 1.95- |
| | | 1.84 (m, 1H), 1.63-1.56 (m, |
| | | 1H), 1.48-1.44 (m, 1H), |
| | | 1.29-1.19 (m, 23H), 0.84- |
| | | 0.79 (m, 3H). |
| 15 | 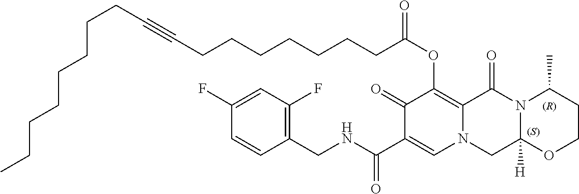 | ¹HNMR (400M, d-DMSO) δ10.15 (t, J = 8 Hz, 1H), 8.65 (s, 1H), 7.38-7.32 (m, 1H), 7.21-7.15 (m,1H), 7.03-6.98 (m, 1H), 5.37- 5.35 (m, 1H), 4.70-4.65 (m, 1H), 4.61-4.56 (m, 1H), 4.50 (d, J = 8 Hz, 1H), 4.41- 4.36 (m, 1H), 3.97-3.91 (m, 1H), 3.84-3.80 (m, 1H), 2.54-2.51 (m, 2H), 2.09- 2.04 (m, 4H), 1.95-1.84 (m, |
| | | 1H), 1.63-1.56 (m, 1H), |
| | | 1.48-1.44 (m, 1H), 1.29- |
| | | 1.19 (m, 25H), 0.84- |
| | | 0.79 (m, 3H). |
| 16 | 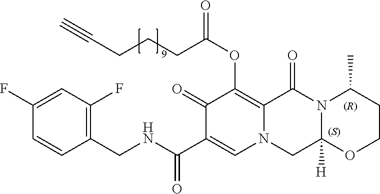 | ¹HNMR (400M, d-DMSO) δ10.15 (t, J = 8 Hz, 1H), 8.65 (s, 1H), 7.38-7.32 (m, 1H), 7.21-7.15 (m, 1H), 7.03-6.98 (m, 1H), 5.37- 5.35 (m, 1H), 4.70-4.65 (m, 1H), 4.61-4.56 (m, 1H), 4.50 (d, J = 8 Hz, 1H), 4.41- 4.36 (m, 1H), 3.97-3.91 (m, 1H), 3.84-3.80 (m, 1H), 2.54-2.51 (m, 2H), 2.09- 2.04 (m, 2H), 1.95-1.84 (m, |
| | | 1H), 1.63-1.56 (m, 1H), |
| | | 1.48-1.44 (m, 1H), 1.29- |
| | | 1.19 (m, 22H). |
| 17 | 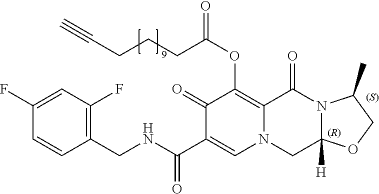 | ¹HNMR (400M, d-DMSO) δ10.18 (t, J = 8 Hz, 1H), 7.93 (s, 1H), 7.34-7.28 (m, 1H), 7.20-7.18 (m, 1H), 7.03-7.01 (m, 1H), 5.37-5.35 (m, 1H), 4.70- 4.65 (m, 1H), 4.61-4.56 (m, 1H), 4.50 (d, J = 8 Hz, 1H), 4.41-4.36 (m, 1H), 3.97- 3.91 (m, 1H), 3.84-3.80 (m, 1H), 2.54-2.51 (m, 2H), 2.09-2.04 (m, 2H), 1.95- |
| | | 1.84 (m, 1H), 1.63-1.56 (m, |
| | | 1H), 1.48-1.44 (m, 1H), |
| | | 1.29-1.19 (m, 2H) |
| 18 | 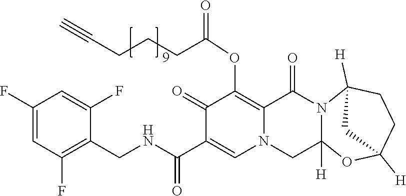 | ¹HNMR (400M, d-DMSO) δ10.23 (t, J = 8 Hz, 1H), 8.08 (s, 1H), 6.98-6.88 (m, 2H), 5.37-5.35 (m, 1H), 4.70- 4.65 (m, 1H), 4.61-4.56 (m, 1H), 4.50 (d, J = 8 Hz, 1H), 4.41-4.36 (m, 1H), 3.97- 3.91 (m, 1H), 3.84-3.80 (m, 1H), 2.54-2.51 (m, 2H), 2.09-2.04 (m, 2H), 1.95- 1.84 (m, 1H), 1.63-1.56 (m, 1H), 1.48-1.44 (m, 1H), |
| | | 1.29-1.19 (m, 22H). |
| 19 | 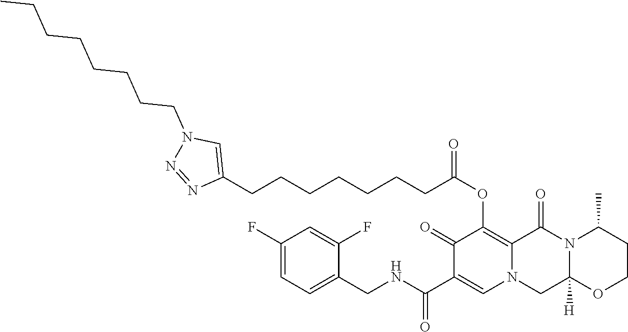 [SMILES: CCCCCCCCn1cc(CCCCCCCC(=O)Oc2c3n(cc(C(=O)NCc4ccc(F)cc4F)c2=O)C[C@@H]2OCC[C@@H](C)N2C3=O)nn1] | ¹HNMR (400M, d-DMSO) δ 10.17 (t, J = 8 Hz, 1H), 8.45 (s, 1H), 7.33-7.25 (m, 1H), 6.81-6.73 (m, 2H), 5.21 (t, J = 4.0 Hz, 1H), 4.90 (t, J = 4.0 Hz, 1H), 4.58 (s, 2H), 4.32-4.28 (m, 3H), 4.17-4.12 (m, 1H), 3.95-3.93 (m, 2H), 2.71- 2.67 (m, 3H), 2.17-2.12 (m, 1H), 1.89-1.84 (m, 3H), 1.79-1.72 (m, 2H), 1.68- 1.66 (m, 2H), 1.51-1.42 (m, 1H), 1.42-1.22 (m, 18H), 1.14-1.06 (m, 2H), 0.86- 0.83 (m, 3H). |
| 20 | 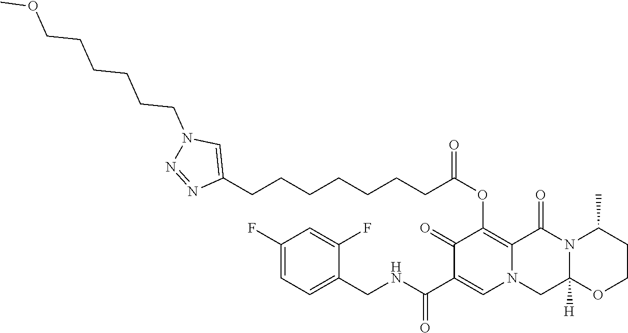 [SMILES: COCCCCCCn1cc(CCCCCCCC(=O)Oc2c3n(cc(C(=O)NCc4ccc(F)cc4F)c2=O)C[C@@H]2OCC[C@@H](C)N2C3=O)nn1] | ¹HNMR (400M, d-DMSO) δ 10.17 (t, J = 8 Hz, 1H), 8.45 (s, 1H), 7.33-7.25 (m, 1H), 6.81-6.73 (m, 2H), 5.21 (t, J = 4.0 Hz, 1H), 4.90 (t, J = 4.0 Hz, 1H), 4.58 (s, 2H), 4.32-4.28 (m, 3H), 4.17-4.12 (m, 1H), 3.95-3.93 (m, 2H),3.45- 3.41 (m, 2H), 3.18 (s, 3H), 2.71-2.67 (m, 3H), 2.17- 2.12 (m, 1H), 1.89-1.84 (m, 3H), 1.68-1.66 (m, 2H), 1.51-1.42 (m, 1H), 1.42- 1.22 (m, 17H), 1.14- 1.06 (m, 2H). |
| 21 | 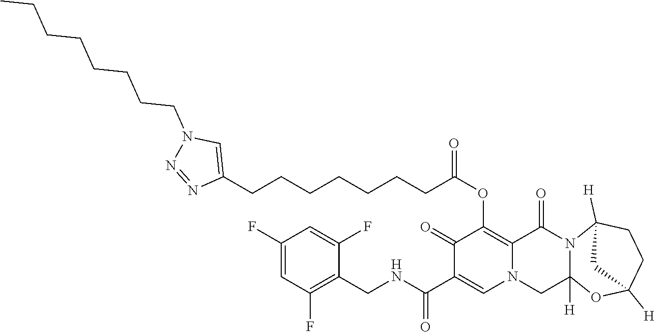 [SMILES: CCCCCCCCn1cc(CCCCCCCC(=O)Oc2c3n(cc(C(=O)NCc4c(F)cc(F)cc4F)c2=O)CC2O[C@@H]4CC[C@@H](C4)N2C3=O)nn1] | ¹HNMR (400M d-DMSO) δ10.23 (t, J = 8 Hz, 1H), 8.45 (s, 1H), 8.08 (s, 1H), 6.98-6.88 (m, 2H), 5.37- 5.35 (m, 1H), 4.70-4.65 (m, 1H), 4.61-4.56 (m, 1H), 4.50 (d, J = 8 Hz, 1H), 4.41- 4.36 (m, 1H), 4.17-4.12 (m, 2H), 3.97-3.91 (m, 1H), 3.84-3.80 (m, 1H), 2.54- 2.51 (m, 2H), 2.09-2.04 (m, 3H), 1.95-1.84 (m, 1H), 1.63-1.56 (m, 1H), 1.48- 1.44 (m, 23H), 1.14- 1.06 (m, 2H), 0.86-0.83 (m, 3H). |
| 22 | 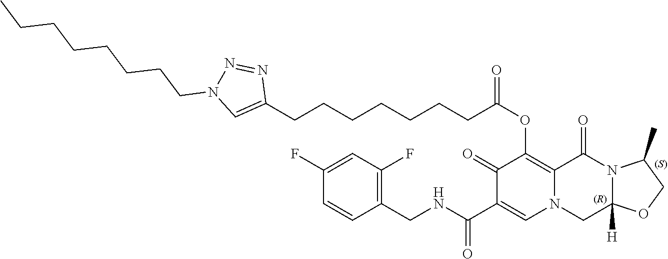 | ¹HNMR (400M, d-DMSO) δ10.18 (t, J = 8 Hz, 1H), 7.93 (s, 1H), 7.34-7.28 (m, 1H), 7.20-7.18 (m, 1H),7.03-7.01 (m, 1H), 5.37-5.35 (m, 1H), 4.70- 4.65 (m, 1H), 4.61-4.56 (m, 1H), 4.50 (d, J = 8 Hz, 1H), 4.41-4.36 (m, 1H), 3.97- 3.91 (m, 1H), 3.84-3.80 (m, 1H), 2.54-2.51 (m, 2H), 2.09-2.04 (m, 2H), 1.89- 1.84 (m, 3H), 1.79-1.72 (m, 2H), 1.68-1.66 (m, 2H), 1.51-1.42 (m, 1H), 1.42- 1.22 (m, 19H), 1.14- |
| | | 1.06 (m, 2H), 0.86-0.83 (m, |
| | | 3H). |
| 23 | 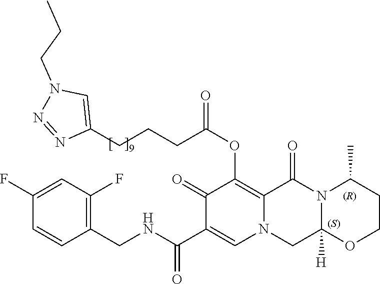 | ¹HNMR (400M, d-DMSO) δ 10.18 (t, J = 8 Hz, 1H), 8.44 (s, 1H), 7.34-7.27 (m, 1H), 6.81-6.73 (m, 2H), 5.21 (t, J = 4.0 Hz, 1H), 4.90 (t, J = 4.0 Hz, 1H), 4.58 (s, 2H), 4.32-4.28 (m, 3H), 4.17-4.12 (m, 1H), 3.95-3.93 (m, 2H), 2.71- 2.67 (m, 3H), 2.17-2.12 (m, 1H), 1.89-1.84 (m, 3H), 1.79-1.72 (m, 2H), 1.68- 1.66 (m, 2H), 1.51-1.42 (m, 1H), 1.46-1.22 (m, 18H), 1.14-1.06 (m, 2H), 0.88- 0.83 (m, 3H). |
| 24 | 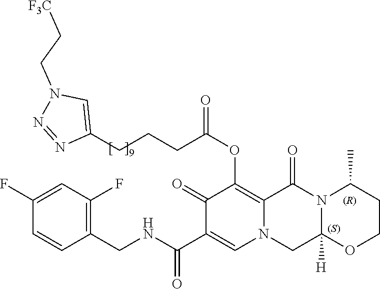 | ¹HNMR (400M, d-DMSO) δ 10.18 (t, J = 8 Hz, 1H), 8.44 (s, 1H), 7.34-7.27 (m, 1H), 6.81-6.73 (m, 2H), 5.21 (t, J = 4.0 Hz, 1H), 4.90 (t, J = 4.0 Hz, 1H), 4.58 (s, 2H), 4.32-4.28 (m, 3H), 4.17-4.12 (m, 1H), 3.95-3.93 (m, 2H), 2.71- 2.64 (m, 5H), 2.17-2.12 (m, 1H), 1.89-1.84 (m, 3H), 1.79-1.72 (m, 2H), 1.68- 1.66 (m, 2H), 1.51-1.42 (m, 1H), 1.46-1.22 (m, 18H). |
| 25 | 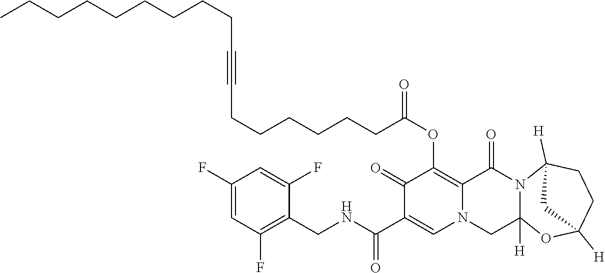 [SMILES: CCCCCCCCCC#CCCCCCCC(=O)Oc1c2n(cc(C(=O)NCc3c(F)cc(F)cc3F)c1=O)CC1O[C@@H]3CC[C@@H](C3)N1C2=O] | ¹HNMR (400M, d-DMSO) δ10.25 (t, J = 8 Hz, 1H), 8.07 (s, 1H), 6.98-6.88 (m, 2H), 5.37-5.35 (m, 1H), 4.70- 4.65 (m, 1H), 4.61-4.56 (m, 1H), 4.50 (d, J = 8 Hz, 1H), 4.41-4.36 (m, 1H), 3.97- 3.91 (m, 1H), 3.84-3.80 (m, 1H), 2.54-2.51 (m, 2H), 2.09-2.04 (m, 3H), 1.95- 1.84 (m, 1H), 1.63-1.56 (m, 1H), 1.48-1.44 (m, 1H), 1.29-1.10 (m, 23H), 0.84- 0.79 (m, 3H). |
| 26 | 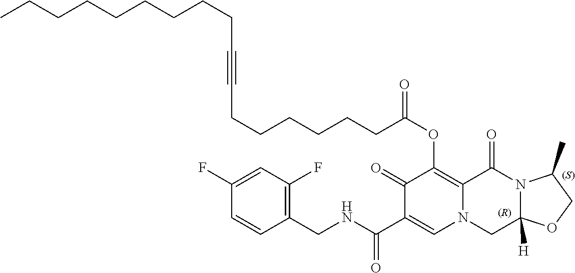 | ¹HNMR (400M, d-DMSO) δ10.18 (t, J = 8 Hz, 1H), 7.93 (s, 1H), 7.34-7.28 (m, 1H), 7.20-7.18 (m, 1H), 7.03-7.01 (m, 1H), 5.37-5.35 (m, 1H), 4.70- 4.65 (m, 1H), 4.61-4.56 (m, 1H), 4.50 (d, J = 8 Hz, 1H), 4.41-4.36 (m, 1H), 3.97- 3.91 (m, 1H), 3.84-3.80 (m, 1H), 2.54-2.51 (m, 2H), 2.09-2.04 (m, 4H), 1.95- 1.84 (m, 1H), 1.62-1.56 (m, 1H), 1.48-1.44 (m, 1H), 1.29-1.10 (m, 23H), 0.84- 0.79 (m, 3H). |
| 27 | 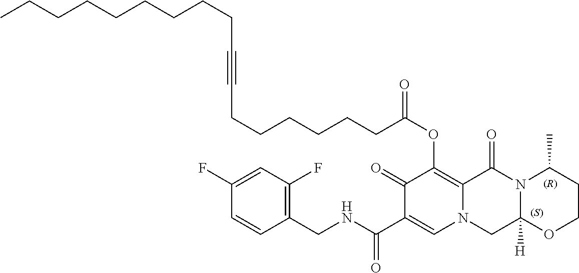 | ¹HNMR (400M, d-DMSO) δ10.15 (t, J = 8 Hz, 1H), 8.65 (s, 1H), 7.38-7.32 (m, 1H), 7.21-7.15 (m,1H), 7.03-6.98 (m, 1H), 5.37- 5.35 (m, 1H), 4.70-4.65 (m, 1H), 4.61-4.56 (m, 1H), 4.50 (d, J = 8 Hz, 1H), 4.41- 4.36 (m, 1H), 3.97-3.91 (m, 1H), 3.84-3.80 (m, 1H), 2.54-2.51 (m, 2H), 2.09- 2.04 (m, 4H), 1.95-1.84 (m, 1H), 1.63-1.56 (m, 1H), 1.48-1.44 (m, 1H), 1.22- 1.09 (m, 25H), 0.82- 0.79 (m, 3H). |

### Example 3 Pharmacokinetic Studies In Vivo

The compounds of Formula I of the present disclosure are metabolized in vivo to the corresponding active ingredients: dolutegravir (DTG), cabotegravir (CAB) and bictegravir (BTG).

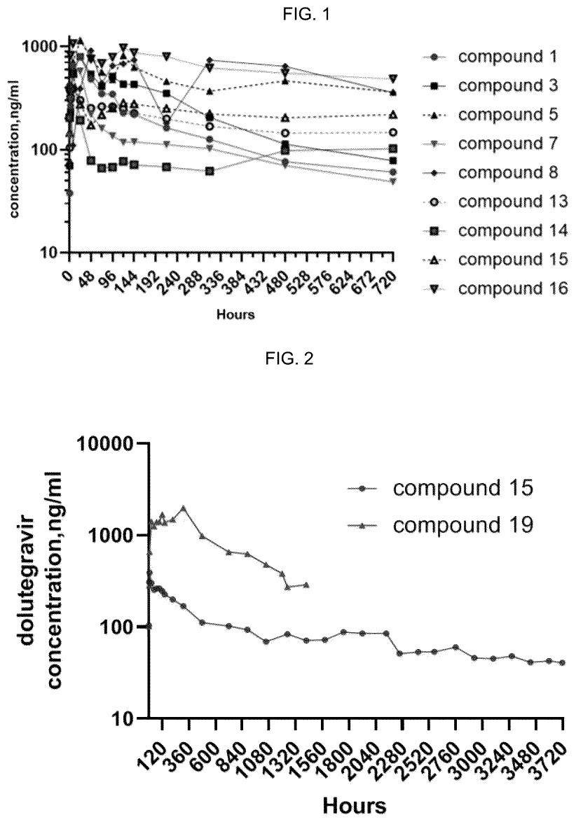

As shown in FIG. 1, after a single subcutaneous injection of Compounds 1, 3, 5, 7, 8, 13, 14, 15, and 16 (administered dose: equivalent to 18 mg/kg of the active ingredient) in rats, mean plasma concentrations of the corresponding active ingredient (DTG or CAB or BTG) versus time were measured.

As shown in FIG. 2, after a single subcutaneous injection of Compound 15 (dosage: equivalent to 18 mg/kg of dolutegravir) and Compound 19 (dosage: equivalent to 45 mg/kg of dolutegravir) in rats, mean plasma concentrations of the corresponding active ingredient (dolutegravir) versus time were measured.

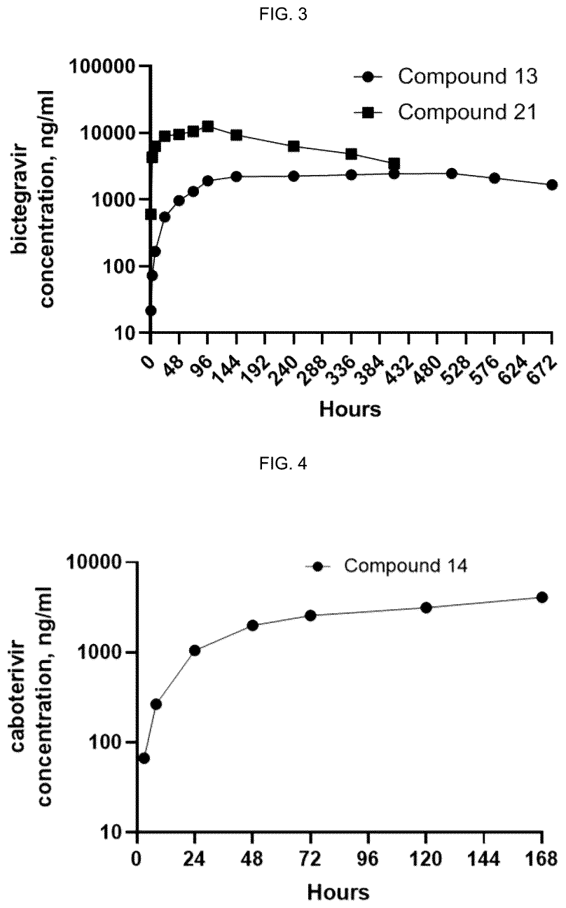

As shown in FIG. 3, after a single subcutaneous injection of Compound 13 and Compound 21 (dosage: equivalent to 18 mg/kg of bictegravir) in rats, mean plasma concentrations of the corresponding active ingredient (bictegravir) versus time were measured.

As shown in FIG. 4, after a single subcutaneous injection of Compound 14 (dosage: equivalent to 18 mg/kg of cabotegravir) in rats, mean plasma concentrations of the corresponding active ingredient (cabotegravir) versus time were measured.

The compounds of the present disclosure have long-acting properties and show the potential to be administered monthly, every two months, every six months, or even every twelve months.

What is claimed is:

1. A compound of Formula I, stereoisomers, pharmaceutically acceptable salts and deuterated compounds thereof: 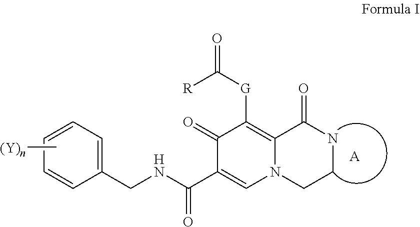
wherein,
A is selected from the group consisting of 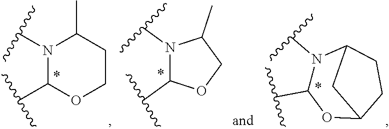
wherein * represents an R or S configuration;
n is 0, 1, 2 or 3;
G is independently selected from oxygen or Gr;
wherein Gr is 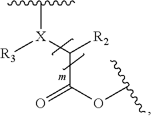
wherein R₂ and R₃ are each independently selected from the group consisting of hydrogen, hydroxy, cyano, C₁₋₆ alkyl, C₆₋₁₀ aryl, C₃₋₁₀ cycloalkyl, arylheterocyclyl, C₁₋₆alkyl-C₆₋₁₀ aryl, C₁₋₆ alkyl-arylheterocyclyl and C₁₋₆ alkyl-C₃₋₁₀ cycloalkyl;
or R₂ and R₃ may be combined with an atom to which they are attached to form a 4- to 10-membered heterocyclyl; wherein heterocyclyl may be substituted with 1 to 4 R₄;
R₄ is independently selected from the group consisting of hydrogen, halogen, C₁₋₆ alkyl and alkoxy;
or any two R₄ may be combined with an atom to which they are attached to form a C₃₋₁₀ cycloalkyl; wherein cycloalkyl may be substituted with 1 to 4 hydrogen, halogen or C₁₋₆ alkyl;
X is selected from the group consisting of oxygen and nitrogen;
Y is halogen;
m is 0 to 20;
R is independently selected from the group consisting of C₂₋₃₀ saturated or unsaturated hydrocarbyl and aliphatic group, wherein hydrocarbyl and aliphatic group may be optionally substituted with at least one heteroatom selected from the group consisting of N, O and S;
R may be optionally substituted with a substituent selected from the group consisting of halo, carboxy, sulfonyl, —S═O, —S(═O)₂, aryl, heteroaryl, C₃₋₁₀ saturated or unsaturated carbocycle and C₃₋₁₀ heterocyclyl, wherein the aryl, heteroaryl, carbocycle and heterocyclyl were optionally substituted with H, C₁₋₂₀ alkyl, C₂₋₂₀ alkenyl, C₂₋₂₀ alkynyl, C₁₋₂₀ haloalkyl, C₂₋₂₀ haloalkenyl, C₂₋₂₀ haloalkynyl, C₀₋₂₀ alkyl-C₆₋₁₀ aryl, heterocyclyl, C₀₋₂₀ alkyl-C₃₋₁₀ cycloalkyl, C₀₋₂₀ alkyl-O— C₁₋₆ alkyl and C₀₋₂₀ alkyl-O— C₁₋₆ haloalkyl.

2. The compound of claim 1, stereoisomers, pharmaceutically acceptable salts or deuterated compounds thereof, wherein
the G is independently selected from oxygen or Gr;
wherein the Gr is 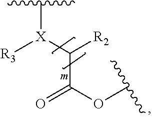
the R₂ and the R₃ are each independently selected from the group consisting of hydrogen, C₁₋₆ alkyl, arylheterocyclyl, C₁₋₆ alkyl-arylheterocyclyl;
or the R₂ and the R₃ may be combined with an atom to which they are attached to form a 4- to 10-membered heterocyclyl; wherein heterocyclyl may be substituted with 1 to 4 the R₄;
the R₄ is independently selected from the group consisting of hydrogen, halogen, C₁₋₆ alkyl and alkoxy;
or any two R₄ may be combined with an atom to which they are attached to form a C₃₋₁₀ cycloalkyl; wherein cycloalkyl may be substituted with 1 to 4 hydrogen, halogen or C₁₋₆ alkyl;
the R is independently selected from the group consisting of C₂₋₃₀ saturated or unsaturated hydrocarbyl and aliphatic group, wherein hydrocarbyl and aliphatic group may be optionally substituted with at least one heteroatom selected from the group consisting of N, O and S;
the R may be optionally substituted with a substituent selected from the group consisting of halo, aryl, heteroaryl, C₃₋₁₀ saturated or unsaturated carbocycle and C₃₋₁₀ heterocyclyl, wherein the aryl, heteroaryl, C₃₋₁₀ saturated or unsaturated carbocycle and C₃₋₁₀ heterocyclyl were optionally substituted with H, C₁₋₂₀ alkyl, C₂₋₂₀ alkenyl, C₂₋₂₀ alkynyl, C₁₋₂₀ haloalkyl, C₂₋₂₀ haloalkenyl, and C₂₋₂₀ haloalkynyl.

3. The compound of claim 2, stereoisomers, pharmaceutically acceptable salts or deuterated compounds thereof, wherein
the G is 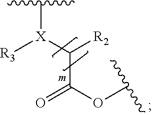
the R is independently selected from the group consisting of C₂₋₃₀ saturated or unsaturated hydrocarby;
preferably, the R may be optionally substituted with a substituent selected from the group consisting of halo, aryl, heteroaryl, C₃₋₁₀ saturated or unsaturated carbocycle and C₃₋₁₀ heterocyclyl, wherein the aryl, heteroaryl, C₃₋₁₀ saturated or unsaturated carbocycle and C₃₋₁₀ heterocyclyl were optionally substituted with H, C₁₋₂₀ alkyl, C₂₋₂₀ alkenyl, C₂₋₂₀ alkynyl, C₁₋₂₀ haloalkyl, C₂₋₂₀ haloalkenyl, and C₂₋₂₀ haloalkynyl.

4. The compound of claim 1, stereoisomers, pharmaceutically acceptable salts or deuterated compounds thereof, wherein
the G is oxygen;
preferably, the R is a heterocycloalkyl having a heteroatom substituted with C₁₀₋₂₀ alkyl-C(O)—; preferably, the R is a heterocycloalkyl having a heteroatom substituted with C₁₅₋₁₈ alkyl-C(O)—; preferably, the R is a heterocycloalkyl having a heteroatom substituted with C₁₅ linear alkyl-C(O)—; wherein, the heteroatom in the heterocycloalkyl being an N atom; preferably, the heterocycloalkyl is a four-membered heterocycloalkyl or a five-membered heterocycloalkyl, and carbon atoms in the heterocycloalkyl are preferably substituted with H or halogen atoms; further, preferably, the R is selected from any one or more of 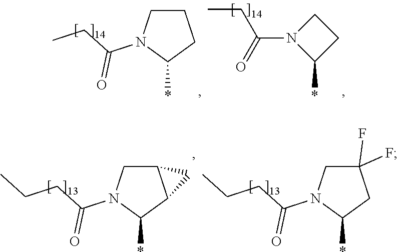
or the R is a saturated C₁₀₋₂₀ alkyl, preferably a saturated C₁₅₋₁₈ alkyl, wherein one or more methylene groups are replaced by —O—; further, preferably, the R is 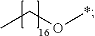
or the R is a C₁₀₋₂₀ linear alkyl with an end group substituted by an alkynyl or a carboxyl; preferably, the R is a C₁₁₋₁₆ linear alkyl with an end group substituted by an alkynyl or a carboxyl; further, preferably, the R is selected from

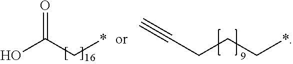

5. The compound of claim 2, stereoisomers, pharmaceutically acceptable salts or deuterated compounds thereof, wherein
the G is 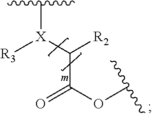
the R is independently selected from the group consisting of unsaturated hydrocarby and C₀₋₁₈ alky-heteroaryl-C₀₋₁₈ alky;
preferably, the R may be optionally substituted with a substituent selected from the group consisting of halo, aryl, heteroaryl, C₃₋₁₀ saturated or unsaturated carbocycle and C₃₋₁₀ heterocyclyl.

6. The compound of claim 5, stereoisomers, pharmaceutically acceptable salts or deuterated compounds thereof, wherein the R is independently selected from the group consisting of —C₁₋₂₀ alkyl-C≡C—C₀₋₂₀ alkyl; preferably, the R is independently selected from the group consisting of —C₅₋₁₀ alkyl-C≡C—C₅₋₁₀ alkyl; preferably, the R is independently selected from the group consisting of —C₆₋₇ alkyl-C≡C—C₅₋₉ alkyl; wherein the alkyl was optionally substituted with halo.

7. The compound of claim 5, stereoisomers, pharmaceutically acceptable salts or deuterated compounds thereof, wherein the R is independently selected from the group consisting of —C₀₋₁₈ alkyl-heteroaryl-C₀₋₁₈ alkyl; preferably, the R is independently selected from the group consisting of —C₅₋₁₂ alkyl-heteroaryl-C₃₋₁₀ alkyl; preferably, the R is independently selected from the group consisting of —C₇₋₁₁ alkyl-heteroaryl-C₃₋₈ alkyl; wherein the alkyl was optionally substituted with halo.

8. The compound of claim 7, stereoisomers, pharmaceutically acceptable salts or deuterated compounds thereof, wherein the heteroaryl is

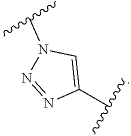 [SMILES: *c1cn(*)nn1]

9. The compound of Formula I, stereoisomers, pharmaceutically acceptable salts and deuterated compounds thereof of claim 1, wherein the compound of Formula I is selected from one of the following structures:

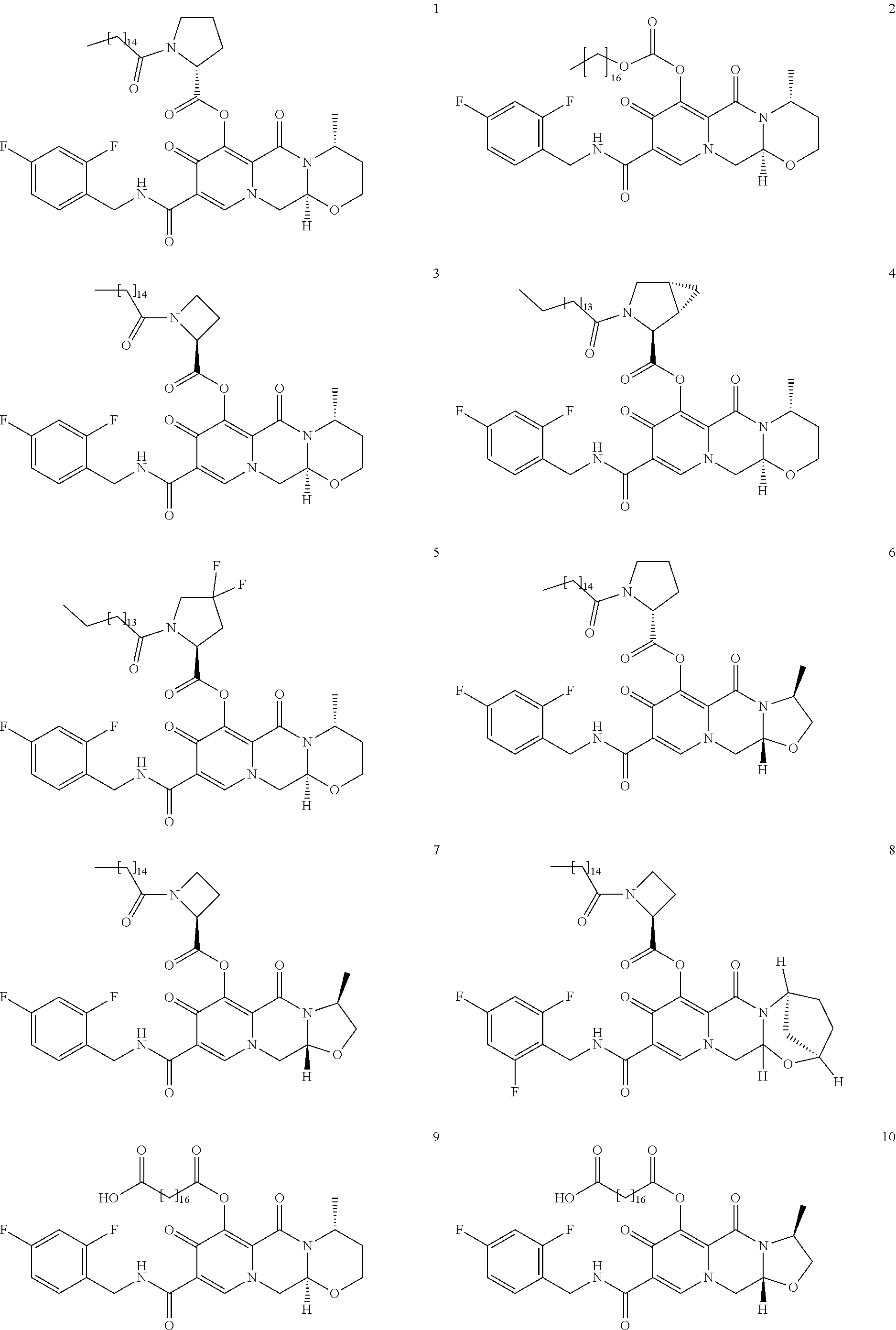

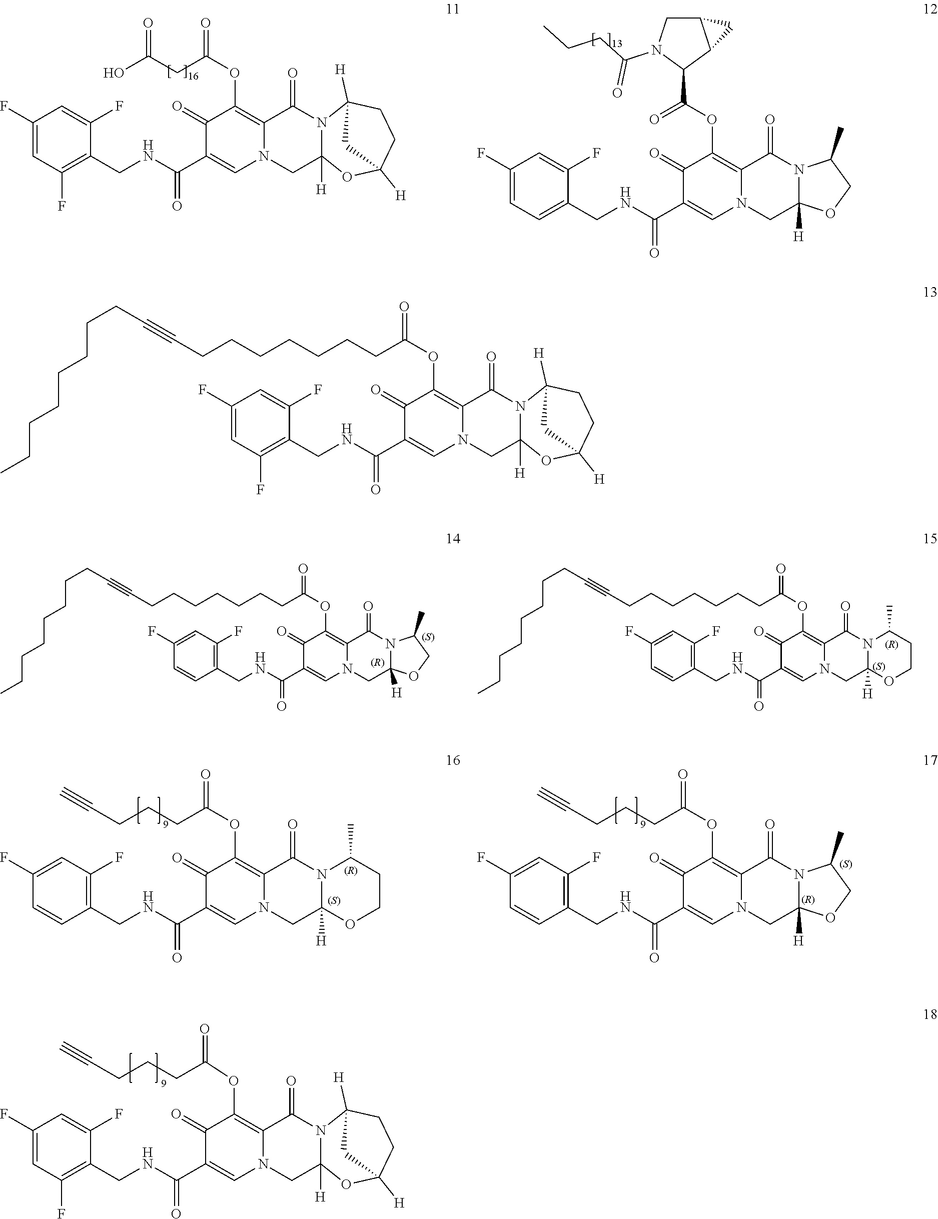

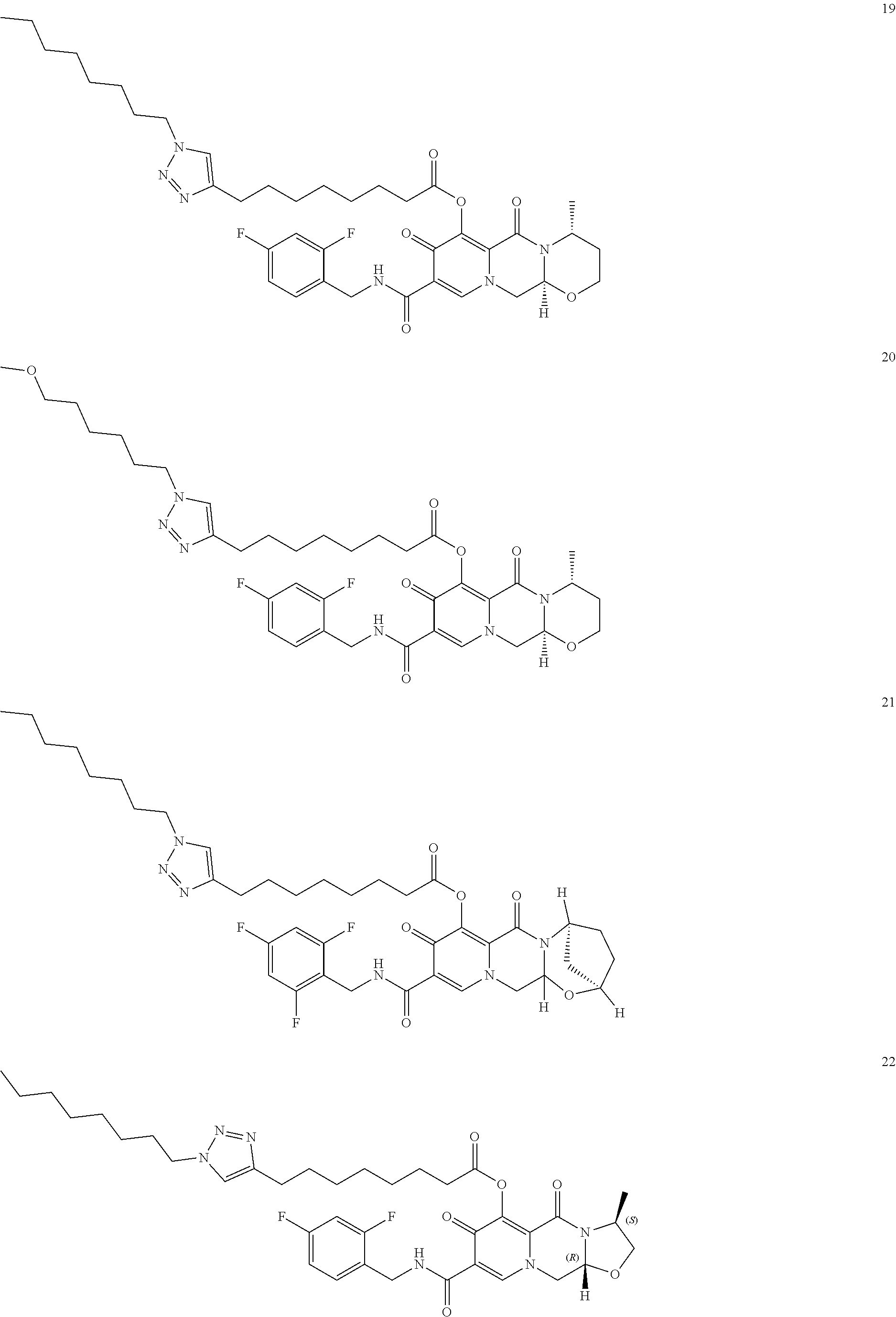

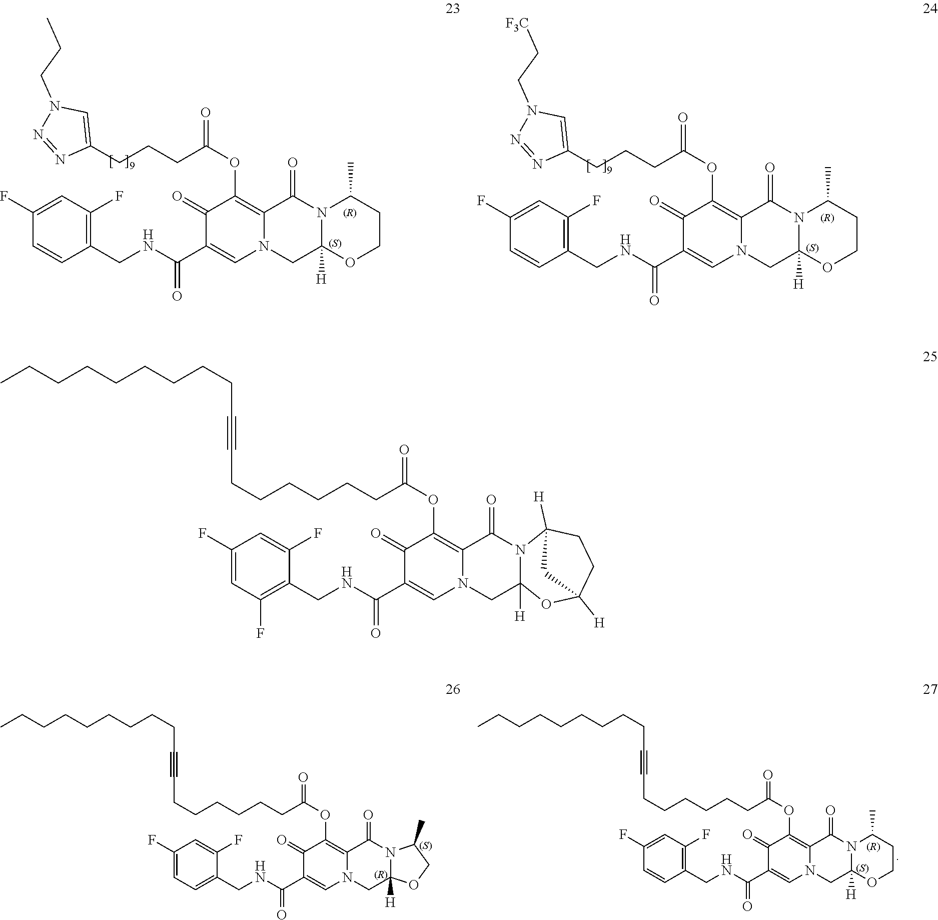

10. A pharmaceutical composition, comprising the compound of claim 1, stereoisomers, pharmaceutically acceptable salts or deuterated compounds thereof, and a pharmaceutically acceptable excipient.

11. A method for treating, inhibiting and/or preventing a disease or disorder in a subject in need thereof, comprising administering an effective amount of the compound of Formula I, a stereoisomer, a pharmaceutically acceptable salt or a deuterated compound thereof of claim 1 to the subject.

12. The method of claim 11, wherein the disease or disorder is a viral infection.

13. The method of claim 12, wherein the viral infection is an HIV infection.

14. The compound of claim 2, stereoisomers, pharmaceutically acceptable salts or deuterated compounds thereof, wherein
the G is oxygen;
preferably, the R is a heterocycloalkyl having a heteroatom substituted with C₁₀₋₂₀ alkyl-C(O)—; preferably, the R is a heterocycloalkyl having a heteroatom substituted with C₁₅₋₁₈ alkyl-C(O)—; preferably, the R is a heterocycloalkyl having a heteroatom substituted with C₁₅ linear alkyl-C(O)—; wherein, the heteroatom in the heterocycloalkyl being an N atom; preferably, the heterocycloalkyl is a four-membered heterocycloalkyl or a five-membered heterocycloalkyl, and carbon atoms in the heterocycloalkyl are preferably substituted with H or halogen atoms; further, preferably, the R is selected from any one or more of 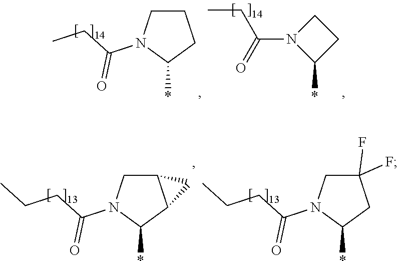
or the R is a saturated C₁₀₋₂₀ alkyl, preferably a saturated C₁₅₋₁₈ alkyl, wherein one or more methylene groups are replaced by —O—; further, preferably, the R is 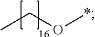
or the R is a C₁₀₋₂₀ linear alkyl with an end group substituted by an alkynyl or a carboxyl;
preferably, the R is a C₁₁₋₁₆ linear alkyl with an end group substituted by an alkynyl or a carboxyl; further, preferably, the R is selected from [chemical structure]
